(19) United States
(12) Patent Application Publication
(10) Pub. No.: US 2023/0332088 A1
(43) Pub. Date: Oct. 19, 2023

(54) METHODS, APPARATUS AND PRODUCTS OF CELL, TISSUE ENGINEERING AND VACCINE/ANTIBODY PRODUCTION SYSTEMS

(71) Applicant: Futrfab Inc., Hague, NY (US)

(72) Inventor: Frederick A. Flitsch, Framingham, MA (US)

(21) Appl. No.: 18/211,264

(22) Filed: Jun. 18, 2023

Related U.S. Application Data
(63) Continuation of application No. 17/617,558, filed on Dec. 8, 2021.
(60) Provisional application No. 62/869,335, filed on Jul. 1, 2019.

(51) Int. Cl. C12M 1/12; A61K 39/00; C07K 14/005; C12M 3/00; C12M 3/06; C12M 1/00; C12N 5/071

(52) U.S. Cl. CPC C12M 37/04; A61K 39/00; C07K 14/005; C12M 21/08; C12M 23/16; C12M 25/14; C12M 29/04; C12M 37/00; C12N 5/0697; C12N 2510/02; C12N 2513/00

(57) ABSTRACT

The present invention provides apparatus and methods for production of tissue structures, organs, vaccines, and antibody products. In some examples, a cleanspace facility may be equipped with fluid interconnections and controls. The fluid interconnections may be located in a primary cleanspace or peripheral to a primary cleanspace. Sterilization may be performed within the primary cleanspace and within the fluid interconnections. In some examples, the facility may include modelling hardware and software, nanotechnology and microelectronic apparatus, and additive manufacturing equipment to print cells and support matrix to allow cells to grow into tissue structures and organs. Novel structures combining various cell types and electronics may be formed with the fabricator. In some examples, advanced vaccine products may be produced entirely within the scalable, sterile, and automated fabricator.

### CROSS REFERENCE TO RELATED APPLICATIONS

This application claims priority to the U.S. Non-Provisional application Ser. No. 17/617,558 filed on Dec. 8, 2021 as a divisional application which in turn claims priority to the United States Patent Cooperation Treaty Application PCT/US20/40377 filed Jun. 30, 2020, as a 371 national phase entry which in turn claims the benefit of the U.S. Provisional Patent Application 62/869,335 filed Jul. 1, 2019. The contents of these heretofore mentioned applications are relied upon and hereby incorporated by reference.

### INCORPORATION BY REFERENCE TO RELATED APPLICATIONS

This application references the U.S. patent application Ser. No. 13/829,212 filed Mar. 14, 2013. This application also references the U.S. patent application Ser. No. 14/988,735 filed Jan. 5, 2016. This application also references the U.S. patent application Ser. No. 14/703,552 filed May 4, 2015, now U.S. Pat. No. 9,263,309 issued Feb. 16, 2016. This application also references the U.S. patent application Ser. No. 14/134,705 filed Dec. 19, 2013, now U.S. Pat. No. 9,159,592 issued Oct. 13, 2015. This application also references the U.S. Provisional Application 61/745,996 filed Dec. 26, 2012. This application also references the U.S. patent application, Ser. No. 14/689,980, filed Apr. 17, 2015. This application also references the U.S. patent application Ser. No. 13/398,371, filed Feb. 16, 2012, now U.S. Pat. No. 9,059,227, issued Jun. 16, 2015. This application also references the U.S. patent application Ser. No. 11/980,850, filed Oct. 31, 2007. This application references the U.S. patent application Ser. No. 11/156,205, filed Jun. 18, 2005, now U.S. Pat. No. 7,513,822, issued Apr. 7, 2009. This application also references the U.S. application Ser. No. 11/520,975, filed Sep. 14, 2006, now U.S. Pat. No. 8,229,585, issued Jul. 24, 2012. This application references the U.S. patent application Ser. No. 11/502,689, filed Aug. 12, 2006, now U.S. Pat. No. 9,339,900 issued May 17, 2016. This application also references the following Provisional Applications: Provisional Application Ser. No. 60/596,343, filed Sep. 18, 2005; and also Provisional Application Ser. No. 60/596,173, filed Sep. 6, 2005; and also Provisional Application, Ser. No. 60/596,099, filed Aug. 31, 2005; and also Provisional Application Ser. No. 60/596,053 filed Aug. 26, 2005; and also Provisional Application Ser. No. 60/596,035 filed Aug. 25, 2005; and also Provisional Application Ser. No. 60/595,935 filed Aug. 18, 2005. The contents of these heretofore mentioned applications are relied upon and hereby incorporated by reference.

### FIELD OF THE INVENTION

The present invention relates to methods and associated apparatus and products which correspond to fabrication systems, processing tools and modeling systems and protocols used to create tissue layers, cell products, vaccine products and antibody products in a cleanspace fabrication environment. Complicated structures based on the production may include products such as organs and functional biomedical apparatus. Arrays of multiple chemical species printing elements or cell printing elements may be combined with microfluidic processors and other techniques to form structures of cells and other materials.

### BACKGROUND OF THE INVENTION

A cleanspace fabricator can create an environment that supports complex material processing in a simple clean environment that is also very sterile. In some examples, people are not located within the primary cleanspace of a cleanspace fabricator. Therefore, their cellular matter, and its associated DNA may be isolated as a contaminant for materials that are being processed in the cleanspace fabricator. There are many different processes that may be performed in a cleanspace fabricator which may benefit from the sterile and clean environment that it affords.

Furthermore, there are numerous types of apparatus that may be created in a cleanspace environment such as the processing of microfluidic processing elements. Microfluidic processing elements may therefore be processed in a cleanspace fabricator and then be used in that cleanspace fabricator to perform processing themselves, leveraging the clean, genetically isolated, and sterile aspects of the environment.

In nature, there are complex structures such as living tissues and organs that could be replicated or produced using technologies that could be efficiently operated within a cleanspace fabricator. The production of living tissues and organs could provide numerous benefits to medical needs of various kinds and to the field of regenerative medicine for example.

A medical environment is an ideal place to study a patient with a medical imaging technique to determine shape, function, and abnormalities about various tissues and organ structures within a patient. The same environment is also an ideal place to extract tissue samples from a patient. A cleanspace facility could be figured to support operations within such a medical environment. In a clean and sterile environment, cells from tissue samples may be isolated and induced to grow into stockpiles of cells.

Cells and cell products as well as other biomaterials may be used in the production of vaccine products and antibody products.

Therefore, it would be very useful to create an environment that is sterile and well controlled, that may house and support equipment for the production of engineered tissues and organs, cell based products and vaccine and antibody products. This may be especially useful if the cell stock that is used for the production of the engineered tissues and organs, or cell products originates from a patient that requires the tissues or organs. Finally, it would also be useful if the information of medical imaging studies may be compiled to created models for the formation of the engineered tissues. Such an infrastructure could be useful for creating novel apparatus based on cells, cell products or vaccine and antibody products.

### SUMMARY OF THE INVENTION

Accordingly, methods and apparatus for a tissue, cell, vaccine or antibody engineering or production system based on these principles are described herein. And the present invention provides apparatus and methods to create tissue layers on substrates, advanced devices including cells and tissue layers for various purposes, cell based products, vaccine products and antibody based products within this engineering system that may be located within a cleanspace fabricator. Massively parallel implementations of chemical species printing elements or cell printing elements may be combined with other techniques to form a tissue processing system or support the other goals.

One general aspect includes a method of forming a tissue layer including configuring a tissue engineering apparatus. The cleanspace fabricator may be configured to process at least a first substrate including tissue layers, where the cleanspace fabricator maintains both a particulate cleanliness as well as a biological sterility cleanliness, where the cleanspace fabricator includes at least a first processing apparatus and a second processing apparatus deployed along a periphery of the cleanspace fabricator, and where the cleanspace fabricator includes automation to move one or more of the first substrate and the first processing apparatus within a primary cleanspace of the cleanspace fabricator. The method also includes having a first toolpod and a second toolPod associated with the cleanspace fabricator, where the first toolpod and second toolpod include at least a first fluid tubing that flows between the first toolpod and second toolpod. The method also includes placing a first sample of cells within the cleanspace fabricator. The method also includes moving a first portion of the sample of cells into a bioreactor (which may also include a bioreactor chamber). The method also includes incubating the cells in the bioreactor. The method also includes flowing a fluid including the first portion of the sample of cells from the bioreactor into a cellular washing system through the first fluid tubing. Other embodiments of this aspect include corresponding computer systems, apparatus, and computer programs recorded on one or more computer storage devices, each configured to perform the actions of the methods.

One general aspect includes a method of forming a tissue layer including configuring a tissue engineering apparatus. The cleanspace fabricator may be configured to process at least a first substrate including tissue layers, where the cleanspace fabricator maintains both a particulate cleanliness as well as a biological sterility cleanliness, where the cleanspace fabricator includes at least a first processing apparatus and a second processing apparatus deployed along a periphery of the cleanspace fabricator, and where the cleanspace fabricator includes automation to move one or more of the first substrate and the first processing apparatus within a primary cleanspace of the cleanspace fabricator, and an interconnection between the first processing apparatus and the second processing apparatus which conducts fluids between at least the first processing apparatus and the second processing apparatus.

Implementations may include one or more of the following features. The tissue engineering apparatus where the interconnection is located proximate to a first tool port of the first processing apparatus and a second tool port of the second processing apparatus where when the first toolpod containing the first processing apparatus and the second toolpod containing the second processing apparatus are advanced into their operating position the interconnection resides at least in part in the primary cleanspace.

The tissue engineering apparatus may further include a means of chemically sterilizing at least a first tube within the interconnection, and a means of sterilizing the tool ports and the interconnection when it is in the primary cleanspace. There may be examples where the means of chemically sterilizing the first tube includes a fluid solution including ozone. There may be examples where the means of chemically sterilizing the first tube includes a fluid solution including chlorine. There may be examples where the means of chemically sterilizing the first tube includes a fluid solution including steam. The tissue engineering apparatus may further include a shroud surrounding the periphery of the first tool port of the first toolpod, the interconnection between the first toolpod and the second toolpod, and the second tool port of the second toolPod. The shrouds may create a sealing surface to a fabricator wall. The tissue engineering apparatus may further include a modelling system, where the modelling system is configured to produce a first digital model which is used to control at least the first processing apparatus, where the first processing apparatus controls equipment to create one or more of a tissue support matrix and a printed deposit of cellular and molecular material. The tissue engineering apparatus may further include a second substrate with a multitude of printing elements arrayed thereupon, where the printing elements are capable of emitting a fluid including at least a first cell to a region within a third processing apparatus based upon a final three-dimensional model. The tissue engineering apparatus may further include a microfluidic processing system to process cellular and chemical material and deliver a product to the printing elements. The method may further include genetically modifying dna of cells of the first sample, where the genetic modification renders the cells to be an omnipotent stem cells. The method may include sorting the omnipotent stem cells from other cells to create a second stock of cells. The method may include examples where the first sample of cells is processed within the microfluidic processing system. The methods may include examples where the microfluidic processing system isolates cells of different cell types. The methods may include examples where the microfluidic processing system performs a genetic modification protocol on at least a cell from the first sample of cells. The methods may include examples where the first sample of cells includes neurons. The methods may include examples where the first sample of cells includes endothelial cells. The methods may include examples where a product of printing the first sample of cells includes continuous capillary vessels. The methods may include examples where a product of printing the first sample of cells includes fenestrated capillary vessels. The methods may include examples where a product of printing the first sample of cells includes discontinuous capillary vessels. The methods may include examples where the first sample of cells includes neurons.

In some examples, the methods may include results where a product of printing the first sample of cells is a data processing device. In other examples, a product of printing the first sample of cells includes a collection of neurons configured to create a feedback loop where an activation or suppression signal is passed to an active element. In still other examples, a product of printing the first sample of cells forms a neuron to electronics electrical interface. The methods may include examples where an intermediate feedback loop signal is processed through a collection of neurons and passed to electronics.

The methods may include examples where the first sample of cells includes myocytes. The methods may include examples where a product of printing the first sample of cells forms a device with a movement capability.

The methods may include examples where multiple samples of cells are processed, where the multiple samples of cells separately include neurons, endothelial cells, and myocytes, and where a product of printing the multiple samples of cells is a cellular device capable of neural processing, movement, and circulatory flow processing.

The methods may further include examples with an electronic circuit where a dendrite or axon of at least a first neuron is in electronic communication with a circuit component of the electronic circuit. Implementations of the described techniques may include hardware, a method or process, or computer software on a computer-accessible medium.

One general aspect includes a method of forming a tissue layer including: configuring a tissue engineering apparatus. The examples of cleanspace fabricators may include those where the cleanspace fabricator is configured to process at least a first substrate including tissue layers, where the cleanspace fabricator maintains both a particulate cleanliness as well as a biological sterility cleanliness, where the cleanspace fabricator includes at least a first processing apparatus and a second processing apparatus deployed along a periphery of the cleanspace fabricator, and where the cleanspace fabricator includes automation to move one or more of the first substrate and the first processing apparatus within a primary cleanspace of the cleanspace fabricator. The method also includes examples where the cleanspace fabricator includes at least a first and a second toolPod. The methods may also include examples where at least a first fluid tubing flows between the first and second toolPod.

The methods also include examples with a modelling system, where the modelling system is configured to produce a first digital model which is used to control at least the first processing apparatus, where the first processing apparatus controls equipment to create one or more of a tissue support matrix and a printed deposit of cellular and molecular material. The method also includes where the first processing apparatus includes a second substrate with a multitude of printing elements arrayed thereupon, where the printing elements are capable of emitting a fluid including at least a first cell to a region within the first processing apparatus based upon a final three-dimensional model. The method also includes where the first processing apparatus further includes a microfluidic processing system to process cellular and chemical material and deliver a product to the printing elements.

The method also includes placing a first sample of cells within the cleanspace fabricator. The method also includes moving a first portion of the sample of cells into a bioreactor. The method also includes incubating the cells in the bioreactor; flowing a fluid including the first portion of the sample of cells from the bioreactor into a cellular washing system through the first fluid tubing; concentrating the sample of cells in a concentrating system; placing the first substrate within the cleanspace fabricator; creating a final digital model, where the final digital model represents a three-dimensional model for depositing of cellular material; forming one or more individual printing system elements; aligning the one or more individual printing system elements in space relative to the first substrate; and printing cells from the concentrated sample of cells upon the first substrate, and using location control signals that are based upon the final digital model.

Implementations may include one or more of the following features. The method further including steps to genetically modify cells of the first sample, where the genetic modification renders the cell to be an omnipotent stem cell; and sorting the omnipotent stem cells from other cells to create a second stock of cells. The methods also include examples where the first sample of cells is processed within the microfluidic processing system. In some examples, the microfluidic processing system isolates cells of different cell types and performs a genetic modification protocol on at least a cell from the first sample of cells.

One general aspect includes configuring a biological processing apparatus, the biological processing apparatus comprising: a cleanspace fabricator, wherein the cleanspace fabricator is configured to process at least a first substrate comprising biological materials, wherein the cleanspace fabricator maintains both a particulate cleanliness as well as a biological sterility cleanliness, wherein the cleanspace fabricator comprises at least a first processing apparatus and a second processing apparatus deployed along a periphery of the cleanspace fabricator, and wherein the cleanspace fabricator comprises fabricator automation to move one or more of the first substrate and the first processing apparatus within a primary cleanspace of the cleanspace fabricator. The biological processing apparatus may also include at least a first toolpod and a second toolpod, wherein the first toolpod and second toolpod comprise at least a first fluid tubing that flows between the first toolPod and second toolPod. The biological processing apparatus also includes a third toolpod comprising a bioreactor, wherein the third toolpod when placed within the cleanspace fabricator occupies a position of one of being above the first toolpod, or being beneath the first toolPod in vertical location. In some examples, the first fluid tubing is connected between the first toolpod and the second toolpod with assistance of the fabricator automation. Some embodiments include a fourth toolPod comprising an input/output station, wherein the input output station comprises a sterilization device to sterilize a material placed into the input/output station, and wherein the fabricator moves the material placed into the input/output station from within the input/output station to within the primary cleanspace. Further examples may also include a fifth toolpod comprising a fill/finish processing equipment; wherein the first toolpod comprises at least a first chromatography column; and wherein the second toolpod comprises at least a second chromatography column. In some examples, the biological process apparatus also includes examples where the bioreactor comprises a genetically modified mammalian cell type, wherein a genetic modification of the genetically modified mammalian cell type encodes for a protein expressed on the surface of a microbe. In some specific examples, the biological processing apparatus may include examples wherein the protein comprises a component of the surface spike protein, and wherein the microbe is SARS-CoV-2.

Implementations may include methods of forming a vaccine product. The method may include the step of configuring a vaccine engineering and production apparatus. The vaccine engineering and production methods include a cleanspace fabricator, wherein the cleanspace fabricator is configured to utilize at least a first substrate comprising a bioreactor, wherein the cleanspace fabricator maintains both a particulate cleanliness as well as a biological sterility cleanliness, wherein the cleanspace fabricator comprises at least a first processing apparatus in a first toolpod and a second processing apparatus in a second toolpod deployed along a periphery of the cleanspace fabricator, and wherein the cleanspace fabricator comprises automation to move one or more of the first substrate and the first processing apparatus within a primary cleanspace of the cleanspace fabricator. The vaccine engineering and production methods include examples wherein the first substrate comprising a bioreactor is moved from within a third toolpod comprising a fabricator input and output function to within the primary cleanspace and then to within the first toolPod. The vaccine engineering and production methods include examples wherein the first substrate further comprises at least a first purification element, at least a first valve, at least a first identification element, and at least a first chemical sensor. The vaccine engineering and production methods include examples wherein the first substrate is a single use element. The vaccine engineering and production methods include placing a first sample comprising either cells or isolated nucleic acid within the cleanspace fabricator and moving a first portion of the first sample into the bioreactor of the first substrate. The vaccine engineering and production methods include flowing a fluid comprising the first portion of the product of the bioreactor from the bioreactor into the first purification element within the first substrate. The vaccine engineering and production methods include collecting an output fluid from processing in the first purification element and moving the output fluid to a fill finish processing equipment in forth toolpod. The vaccine engineering and production methods include packaging the output of the fill finish processing equipment in a sterile container and removing the packaged output from the vaccine engineering and production apparatus.

### BRIEF DESCRIPTION OF THE DRAWINGS

The accompanying drawings, that are incorporated in and constitute a part of this specification, illustrate several embodiments of the invention and, together with the description, serve to explain the principles of the invention:

FIG. 1A—An illustration of a small tool cleanspace fabricator in a sectional type of representation.

FIGS. 1B-1I—Exemplary illustrations of toolPods in concert with tissue engineering cleanspace fabricator examples.

FIGS. 1J-1M—Exemplary illustrations of toolPods, fluid interconnections and fabricator structures in concert with tissue engineering cleanspace fabricator examples.

FIG. 2—An illustration of a full substrate imaging apparatus with highlighted regions illustrated at higher scale to depict a collection of individual imaging elements.

FIGS. 3A-D—Exemplary depictions of an array of imaging elements and a close-up view of an exemplary small sized imaging element.

FIG. 4—A Flow chart depicting exemplary methods of production of an imaging apparatus.

FIG. 5—An exemplary processor that may be useful for some embodiments of imaging systems.

FIG. 6—An exemplary processing flow for printing of cells.

FIG. 7—An alternative exemplary processing flow for the printing of cells.

FIG. 8—An alternative exemplary processing flow for the printing of cells.

FIGS. 9A-D—An exemplary processing flow to produce a kidney organ.

FIG. 10—An exemplary processing flow to produce tissue layers.

FIG. 11—An exemplary tissue engineering cleanspace fabricator with toolPods and fluid interconnections.

FIG. 12—An exemplary vaccine/antibody production cleanspace fabricator with toolPods and fluid interconnections.

FIGS. 13A and 13B—An exemplary single use vaccine production substrate according to the present specification.

### DETAILED DESCRIPTION OF THE INVENTION

In patent disclosures by the same inventive entity, the innovation of the cleanspace fabricator has been described. In place of a cleanroom, fabricators of this type may be constructed with a cleanspace that contains the wafers, typically in containers, and the automation to move the wafers and containers around between ports of tools. The cleanspace may typically be much smaller than the space a typical cleanroom may occupy and may also be envisioned as being turned on its side. In some embodiments, the processing tools may be shrunk which changes the processing environment further.

### Description of a Linear, Vertical Cleanspace Fabricator

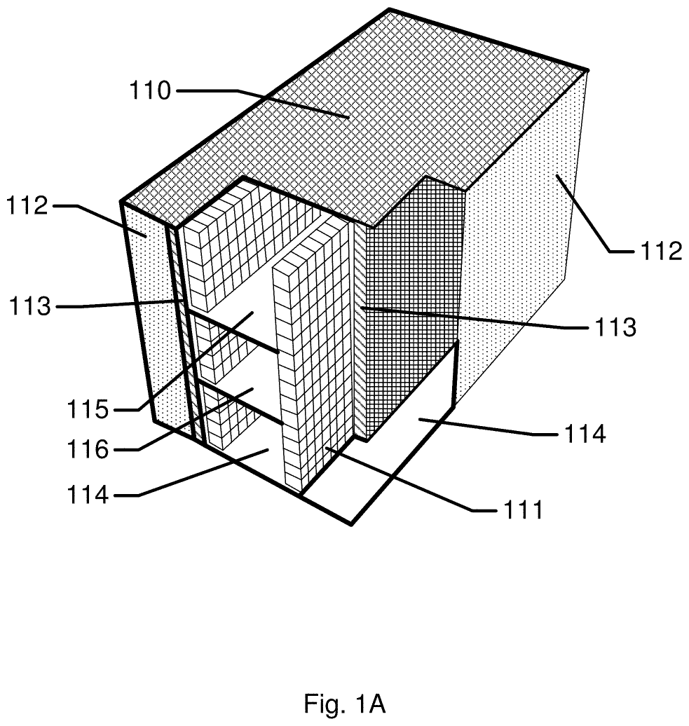

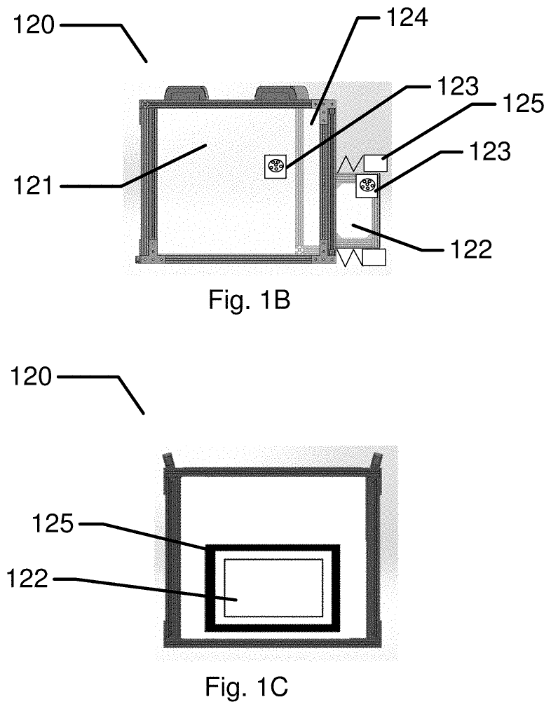

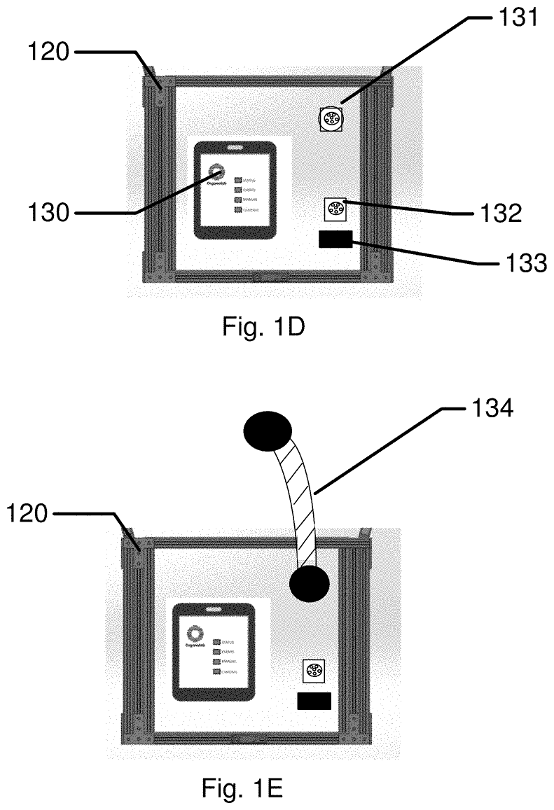

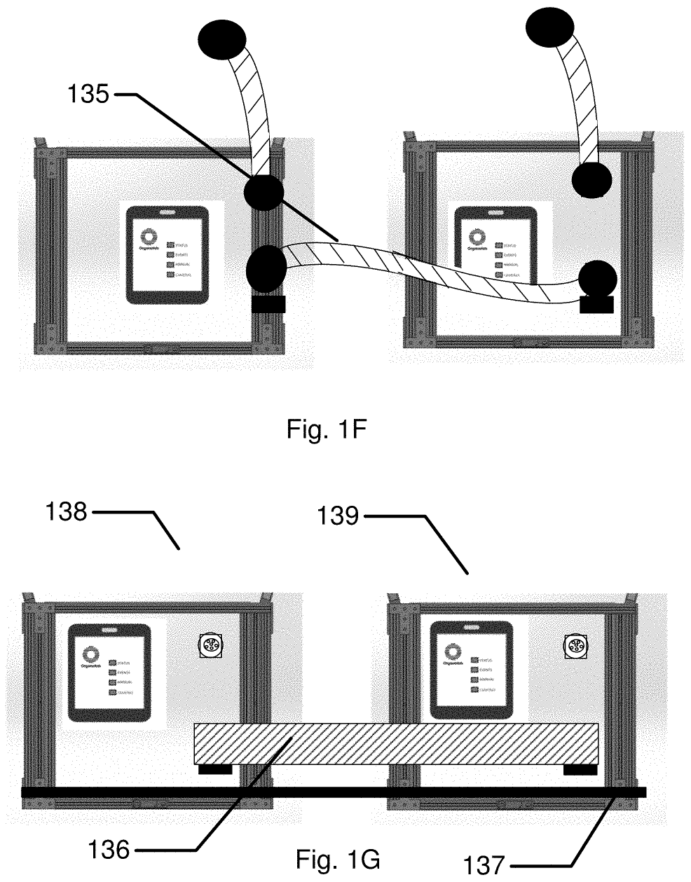

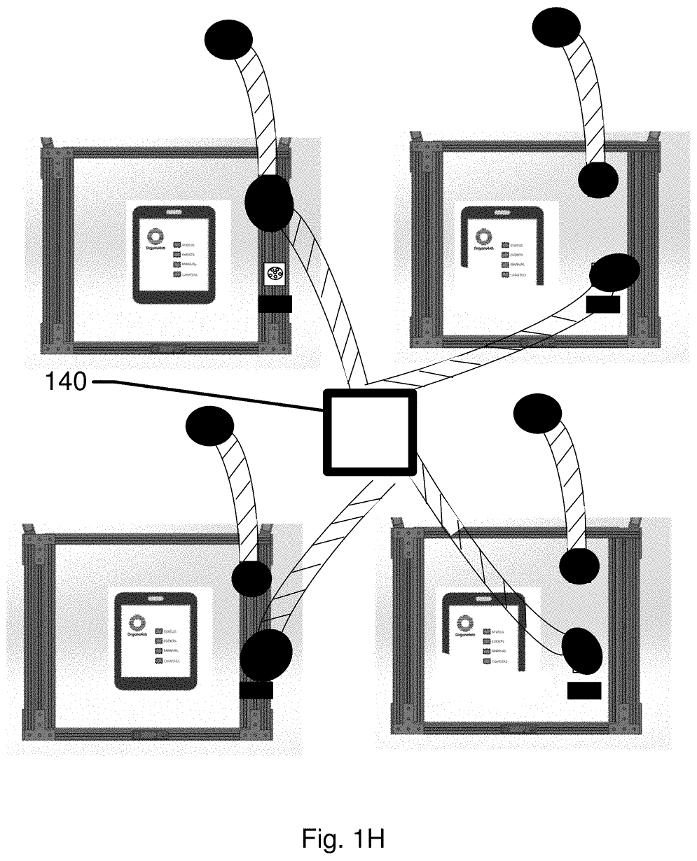

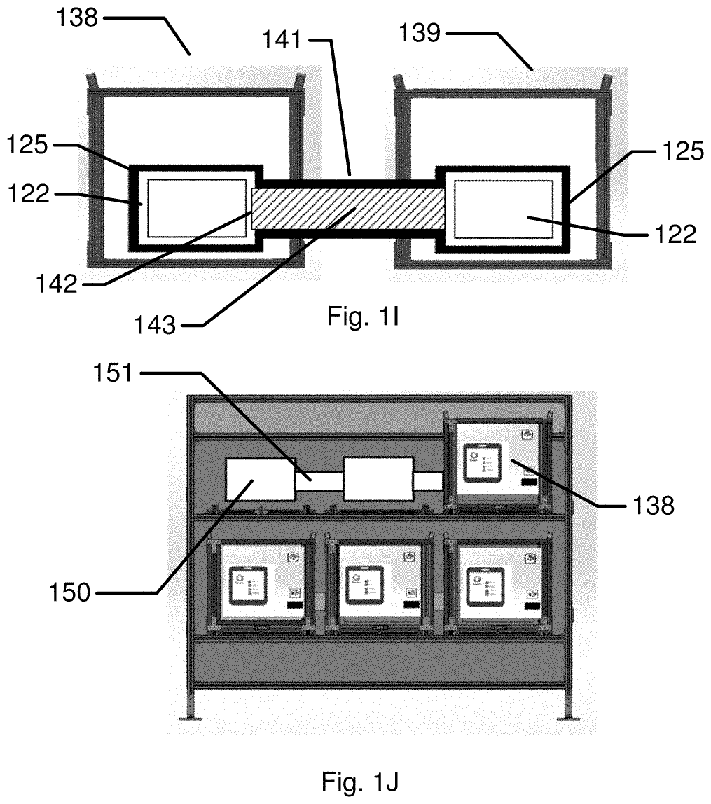

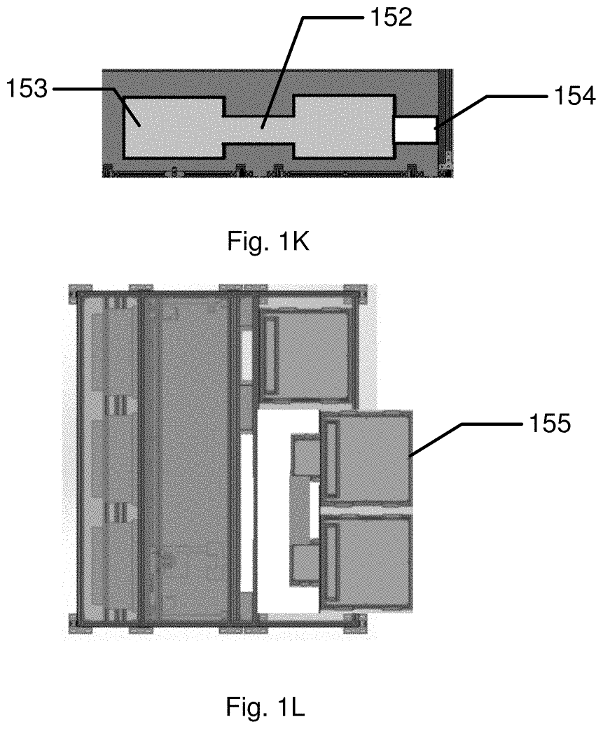

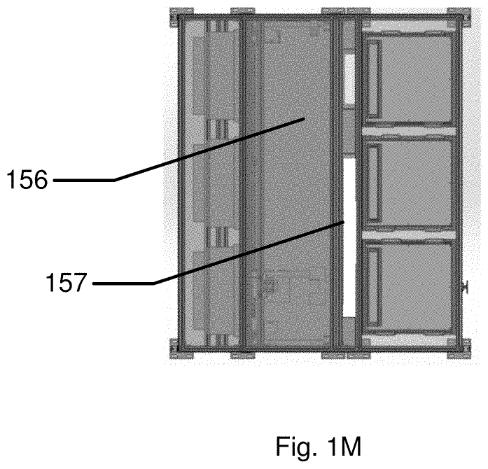

There are a number of types of cleanspace fabricators that may be possible with different orientations. For the purposes of illustration, one exemplary embodiment includes an implementation with a fab shape that is planar with tools oriented in vertical orientations. An exemplary representation of what the internal structure of these types of fabs may look like is shown in a partial cross section representation in FIG. 1A. Item 110 may represent the roof of such a fabricator where some of the roof has been removed to allow for a view into the internal structure. Additionally, items 112 may represent the external walls of the facility which are also removed in part to allow a view into external structure.

In the linear and vertical cleanspace fabricator of FIG. 1A there are a number of aspects that may be observed in the representation. The “rotated and shrunken” cleanspace regions may be observed as cleanspace regions 113. The occurrence of cleanspace regions 113 on the right side of the figure is depicted with a portion of its length cut off to show its rough size in cross section. The cleanspaces lie adjacent to the tool pod locations. Depicted as item 111, the small cubical features represent tooling locations within the fabricator. These locations are located vertically and are adjacent to the cleanspace regions (113). In some embodiments a portion of the tool, the tool port, may protrude into the cleanspace region to interact with the automation that may reside in this region.

Floor 114 may represent the fabricator floor or ground level. On the right side, portions of the fabricator support structure may be removed so that the section may be demonstrated. In between the tools and the cleanspace regions, the location of the floor 114 may represent the region where access is made to place and replace tooling. In some embodiment, as in the one in FIG. 1A, there may be two additional floors that are depicted as items 115 and 116. Other embodiments may have now flooring levels and access to the tools is made either by elevator means or by robotic automation that may be suspended from the ceiling of the fabricator or supported by the ground floor and allow for the automated removal, placement, and replacement of tooling in the fabricator.

Description of a Chassis and a toolPod or a Removable Tool Component

In other patent descriptions of this inventive entity (patent application Ser. No. 11/502,689 which is incorporated in its entirety for reference) description has been made of the nature of the toolPod innovation and the toolPod's chassis innovation. These constructs, which in some embodiments may be ideal for smaller tool form factors, allow for the easy replacement and removal of the processing tools. Fundamentally, the toolPod may represent a portion or an entirety of a processing tool's body. In cases where it may represent a portion, there may be multiple regions of a tool that individually may be removable. During a removal process, the tool may be configured to allow for the disconnection of the toolPod from the fabricator environment, both for aspects of handling of product substrates and for the connection to utilities of a fabricator including gasses, chemicals, electrical interconnections, and communication interconnections to mention a few. The toolPod represents a stand-alone entity that may be shipped from location to location for repair, manufacture, or other purposes.

Referring to FIG. 1B an exemplary front view of a toolPod that may be used in a tissue engineering, cell production and vaccine/antibody fabricator is illustrated in a non-limiting sense. The toolPod 120, may be a stand-alone entity that may contain one or more processing tools and or processing regions within an internal space 121. The toolPod 120 may have a tool port for the transfer of substrates and substrate vessels into and out of the internal space 121 where the processing regions are contained. The toolPod 120 may have one or more interconnection ports 123 that allow for the pass through or coupling of fluidic processing tubes through the toolPod walls. There may be an orifice controlling device 124 which may be used to open and close the tool port to the exterior. In some examples a gate valve may be used, in other examples a film on cylinders may be used to rotate into the orifice an opening or a closed film any device that can open and close a port to an exterior region may be used. In some examples, a shroud 125 may surround the tool port 122 to create a sealing interface as a toolPod is moved into an operating location. Referring to FIG. 1C an end view of the toolPod shows the shroud 125 surrounding the tool port 122. The shroud may have a mating surface that enhances a formation of a seal when given a pressure by advancing the toolPod into the cleanspace.

In some embodiments the toolPod may include a provision to join with other toolPods to provide a connected combination that may allow interconnections between the tools, such as in a non-limiting sense for fluid interconnection. In some examples, the interconnection may include a physical support upon which or inside which fluid connections may be routed between to the tools and in some examples to external input output connections and junction ports. In some examples, single use implementations of the surfaces that interact with cells and other products may be supported by the designs of interconnections between tool pods.

Referring to FIG. 1D, an exemplary back view of a toolPod 120 is illustrated with a number of features. As mentioned previously, the toolPod 120 may have a fluid interconnection port 132 as well as an interconnection support feature 133. The fluid interconnection port 132 may merely be a feedthrough, which may be sealable, that allows fluid tubing to pass from the external spaces of the toolPod to the external. In other examples, tubing from inside the toolPod may be routed up to the interconnection port 131, and the structure of the port may allow for connection to external tubes to be made at interconnection components. In an example a set of barbed fittings may protrude from the interconnection port 132 and may receive one or more tubes, or a collection of tubes that seal to the fittings. Any known type of fitting to create a seal, or process to seal tubes with welding, gluing, pressure fittings and the like may be used. A support structure for the tubing may be connected to the support feature 133.

Continuing with FIG. 1D there are numerous other illustrated features. An interface 130 may be used to present data, pictures, video, and the like to a user. The interface may be connected to an internal data processing device, or it may be incorporated into a data processing device. An I/O device, such as a touch screen interface, may allow a user to may inputs for control decisions, functional control, data entry, and the like. Other I/O systems may be used for the system interface. As well, the data processing capabilities of the toolPod may include wireless communication systems that can present the data to a user and accept input from, such as a smart device of a user. The wireless communication may occur with WiFi, Bluetooth, other near fear communication or with priority communication protocols. The toolPod may interact/communication with the fabricator control systems and may interact with automation control systems of the chassis device upon which the toolPod may sit. In some examples, utilities such as vacuum, electricity, gasses, data communications, exhaust air flows, pressurized air flows and the like may be provided by interconnections made between the toolPod and its corresponding chassis. Referring to FIG. 1E, in some examples an “umbilical cord” 134, which may be a generalized term for these types of interconnections, may be used to connect a number of utility systems from the fabricator to the toolPod. Referring to FIG. 1F, in some examples in addition to an umbilical cord 134, or not shown other means of connecting toolPods to the facility, there may be an interconnection 135 of one or more tubes between two toolPods. Referring to FIG. 1G, two adjacent toolPods 138-139 may be interconnected to become one entity. A connecting plate 137, a physical surface attaching each toolPod to hold them in rigid place, may connect the two toolPods 137-139 and a supporting feature 136 may contain and/or support a tubing bundle which is interfaced to the two toolPods creating connections that reside in the exterior portions of the fabricator. Referring to FIG. 1H, other examples of tube interconnections are illustrated where a coordinating interconnection device 140 may receive tube connections from multiple toolPods and route them to different toolPods. In some examples, these illustrated external bundles of tubes may be installed onto toolPods before the toolPods are installed into a fabricator. In other examples, the external bundles may be added, removed, and replaced while toolPods reside within the fabricator. The coordinating interconnection device 140 may have internal tube components that interconnect one inputted tube with one or more tubes from another bundle of tubes. In some examples, the coordinating interconnection device 140 may have valves internally that may provide for programmable interconnection of tubing inputs.

The interconnection between the tool pods may exist at the tool ports and therefore protrude into the primary cleanspace when the tools are in an operating position. Referring to FIG. 1I, an illustration of making interconnections between toolPods where the interconnections are advanced into the primary cleanspace of the fabricator is illustrated. Two toolPods 138-139 may each have a shroud 125 around a tool port 122 upon which a tubing bundle interconnect is located as well as connection points 142 for interconnecting a support structure 143 that will protrude into the primary cleanspace. Shroud pieces 141 will also surround the placed support structure so that when it along with toolPods are advanced into the fabricator the shroud pieces will form a seal with the wall structures of the fabricator.

In some examples, the tubes of the interconnection may be sterilized in various manners. A chemical solution may be flowed through the tubes of the interconnection to sterilize the internal space. Examples of chemical solutions may include water solutions of ozone, chlorine, soaps as non-limiting examples. Depending on the materials of the tubing interconnections steam may be introduced through the connections for sterilization. The external portions and the junctions of the tubing may be irradiated with UV light or treated in the manners that the external connections were treated, and UV light may be used to provide sterilization of the components surfaces constantly or intermittently.

In other examples, the interconnection may exist in between the tools and reside in a secondary cleanspace where the tool bodies are located when they are in an operating condition. In other examples, the interconnection may be located at the exterior side of the tool bodies which may reside on the periphery of the secondary cleanspace region as described in relationship to FIGS. 1F-1H. In some examples, the secondary cleanspace region may not be cleaned above the ambient level of cleanliness.

In some examples the secondary cleanspace may be an isolated region with doors or pass-throughs that isolate the environment. The secondary cleanspace may include filters above the space or may include horizontal air flow or may allow the airflow from the primary cleanspace to transit into the area before being returned to the air handlers. In some examples, a mobile cleanroom may be used to service locations where tooling is being changed. In examples where multiple toolPods are interconnected, support structures which allow for the placement of toolPod combinations at appropriate locations on a tooling rack may be used.

In some examples the toolPod may include a communications junction box. The communications junction box may take various types of communication and data sources from a variety of tooling devices that may be contained in the pod and convert or coordinate the communications to be standardized to a fab-wide communication protocol allowing for easier incorporation of new tooling into a pod which then correctly communicates with the fab.

In some examples, the toolPod may be divided into multiple tooling locations, where the tooling may be isolated from each other or may be shared in a single space or a partial combination of these.

In some examples, the toolPod may be a base entity that sits upon a chassis of a standard size but allows for different size toolPod surroundings to be included.

In some examples, the chassis units may include motorized control bases that move tools from an operating to an “open” location. In cases where multiple tool pods are interconnected, the motorized chassis elements may be coordinated by a controller to keep the chassis systems aligned.

In some examples, the tool ports of various tool pods may stick into the primary cleanspace. As discussed, the tool ports may include a surrounding shroud that may interact with the wall surrounding the openings into the cleanspace of the fab. The shroud may be spring loaded or otherwise actively adjusted as a toolPod is introduced into the fab, so that a seal may be maintained. In the fab wall there may be actively controlled openings that allow for toolPods to be entered into the fab while the fab air is still isolated. Referring to FIG. 1J a front view of a fabricator with two open locations, that is without toolPods, is illustrated. At the rear of the toolPod space of the fabricator is the wall ceiling the primary cleanspace from the secondary cleanspace. In this wall may be openings that have doors, such as gate valves, that open and close portions of the wall to allow the tool ports of pods and also tubing structures that reside in the primary cleanspace to pass through the wall and into the primary cleanspace. In FIG. 1J, the tool port opening 150 and the tubing interconnection opening 151 are illustrated in a closed position. When there are no tool pods in the locations, these openings 150 and 151 are in a closed position so that the cleanspace air does not leak out of the fabricator space. Referring to FIG. 1K, the tool port opening 153 and the tubing interconnection opening 154 are shown in an open position. When toolPods are being advanced into the fabricator and the shroud pieces form a seal these openings will be moved to open positions so that the structure of the tool port and any attached tubing constructs may pass through the openings 152 while maintaining the integrity of the primary cleanspace.

In some other examples, some or all interconnected tools may not have a need for a tool port for substrate movement. In some of these examples, some of the toolPods may include just an interconnection structure that may move into the primary cleanspace. In some examples, a region of the tool primary cleanspace boundary wall, i.e., where the tool structure or a shroud attached to the tool comes up against a wall, may include closure devices which could be independently controllable to create openings in the return air configuration through which toolPod connected structures such as tool ports and interconnection structures may pass in controlled manners. In general, a toolPod or combination of toolPods may be advanced towards the primary cleanspace wall and a protruding shroud may intersect the wall forming a degree of sealing. Next, a portion of the wall, for example a type of gate valve, may open up, exposing a region for the tool port and interconnect structures (if equipped) to proceed into the primary cleanspace. The portion that opens up may be a combination of a number of gate structures.

Referring to FIG. 1L an illustration of two tool pods with two tool ports with interconnects between them being advanced is illustrated with an initial position 155. Moving into the fabricator results in a seal being formed and places the tool pods and the interconnects into the primary cleanspace. Referring to FIG. 1M the exemplary loading process where a cross section of the wall entities and their respective locations is illustrated after the tools have advanced into their operating position. The ends of the tool ports and tubing supports may protrude into the primary cleanspace 156. The cross-section illustrates a combination 157 of two toolPods and one tubing interconnection to pass through the wall.

In some examples, a toolPod may include a basic set of processing tool components as well as other components. A communications hub which may also include data processing capabilities may be included. Display systems to present status and other data to a user viewing the tool pod may be included. In some examples, display systems may also include interaction for a user such as through a touch screen or through a verbal communication capability. Various imaging devices that can provide video views of various portions of the internal and external portions of the tool pod.

In some examples, a toolPod may include temperature control and regulating aspects that may cool portions of components of a processing tool or may cool the air space of the contents of the tool pod.

In some examples, a toolPod may include filtration systems which may filter air as it is either or both introduced into the toolPod or circulated within the toolPod. In some examples, sterilization devices may be included within a toolPod. In some examples, a sterilization device may include high energy radiation emitters such as UV light or other energetic bands of electromagnetic radiation or particle beam radiation. In some examples, a sterilization device may include chemical emitters, such as in a non-limiting example an ozone emitter or an alcohol misting device. Portions of circulating air may be directed to sterilizing portions of the air circulating loop which may have sterilizing capability which in addition to the other capabilities mentioned above may include heating of the air and/or introduction of steam which may be subsequently cooled before the recirculated air is returned within the toolPod.

In some examples, a power control device may be included with a tool pod or in electrical connection to a tool pod, such as through a chassis. A power control device may also include backup power generation capability in some examples.

In some examples, toolPods may include interface connections for chemical flow into and out of the toolPod. In other examples a connector, which may be termed an “umbilical” cord, may connect to a toolPod from another toolPod or from a toolPod to the facilities of the fabricator itself. The connection may be reversible to allow a toolPod to be connected and disconnected as it is placed into a position in the fabricator The connector may include various connections such as electrical, gasses, chemicals, vacuum, exhaust inflows and exhaust outflows as non-limiting examples. In some examples a single use device may include a connector aspect that functions for an umbilical cord and allows for single time connections to a toolPod.

Combinations of individual fabricators may be added together with ports allowing for materials, components, fluids, and the like to be connected between the versions. In other examples, the fabricators may be scaled to have multiples of the numbers of tool positions as have been described. A fabricator may also be formed from multiple standalone copies of the fabricator units as have been described. In some examples, composite fabricators may be formed from combinations of one or more cleanspace fabricator elements in combination with equipment operating in a cleanroom or standard room configuration.

A toolPod may be supported as a standalone entity upon a toolPod support stand. The toolPod support stand may provide the various interconnections and services that a toolPod may have when placed in a cleanspace fabricator as has been described herein and in reference documents. A standalone toolPod may be set to work in a lab environment, in a test environment, or in a preparatory environment for a production environment. In some examples, research and development on a toolPod's function elements may be performed in a standalone setting. In some other examples the processing of a single toolPod may be performed on a test stand.

### Imaging Apparatus

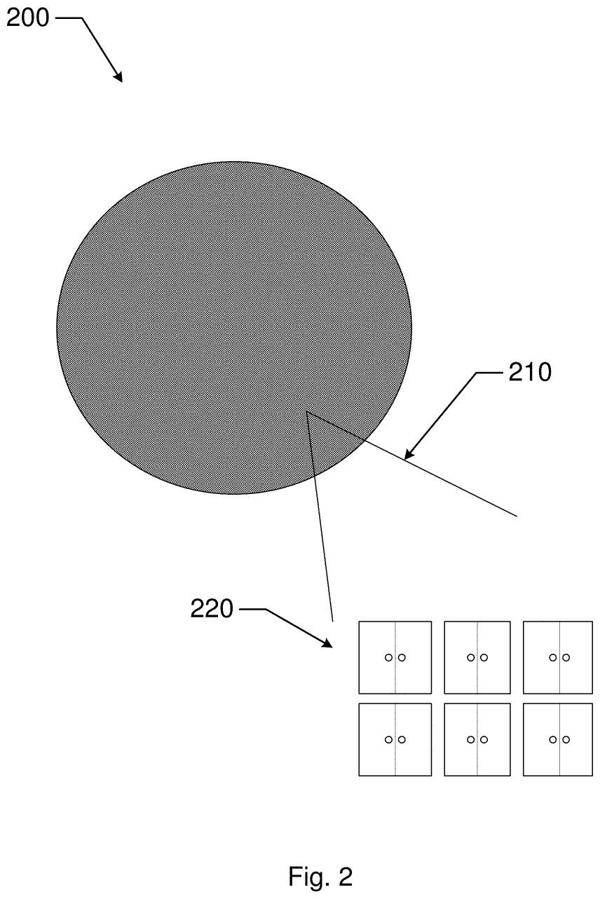

An imaging apparatus of various types may be used in the various cleanspace fabricator designs that have been described herein and in other referenced applications. Referring to FIG. 2 at item 200 an exemplary imaging apparatus in the exemplary form factor of a round substrate is depicted. In some embodiments, the imaging apparatus may be comprised of a large number of similar elements. As shown in a magnified view 210, the individual elements may be arranged in a regular pattern 220.

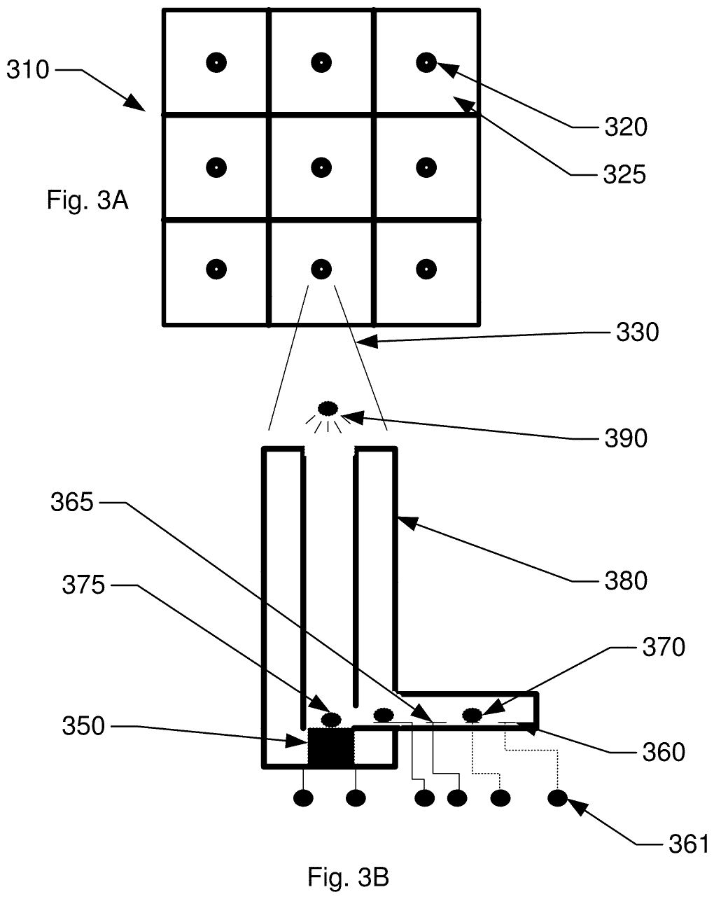

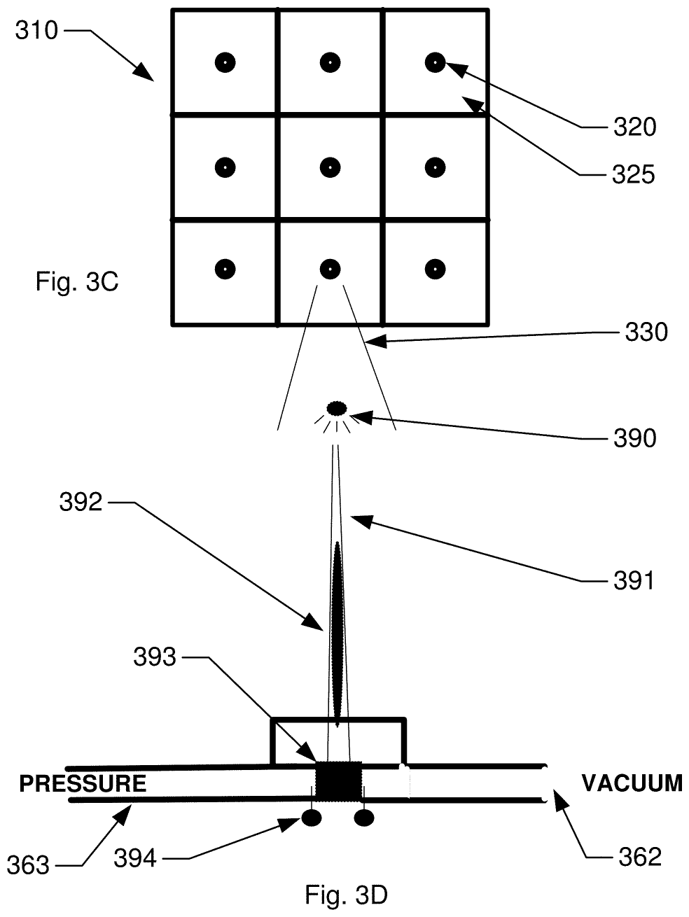

Referring to FIG. 3A at a close up of an imaging element may be depicted in cross section and FIG. 3B a plan view. A type of micro imaging element may be found in reference to FIGS. 3A and 3B. At 3A, item 310, an exemplary array of nine elements such as 325 with an associated image element 320 may be found. One of the elements represented in the close-up 330 of FIG. 3B may be found. This element may be useful for ejecting nanoscale droplets of chemical reactant to react with resist layers to form imaged layers. Item 390 may be an ejected droplet which may contain chemicals, cells or both chemicals and cells. Item 380 may be an element to eject a droplet 375. A piezoelectric element 350 may be useful as such an ejection element or other such features as may be found in ink jet printing technology may be represented by 350. At 370 droplets may be moved by microfluidic techniques through the use of coated electrodes such as items 360 and 365. The electrodes may receive electrical control signals through interconnects from controlling systems. An example of such an electrical connect is depicted at 361.

In some alternative examples, referring to FIGS. 3C and 3D, an array with the same feature aspects such the array 310, element 325 with imaging element 320. In this example, the close-up 330 shows a droplet 390 emerging from a pipet head 391. Pipets can be used to draw up material to be ejected 392. A switch 393, can open the pipet to vacuum 363 to draw material into the pipet and may switch to a pressure 362 situation under activation from electrical contacts 394. The illustration shows an array of 9 elements, however much larger arrays may be built. The pipets may be located into reservoirs containing the material to be distributed. Large channels may receive numerous pipets simultaneously. The pipets may collect a small enough volume of material that a single cell may occupy the pipet. In some examples, an optical detection system may observe the droplet in the pipet to determine the presence of a single cell in the pipette. In some examples, the pipette reservoir may be filled from an external port connecting to the reservoir of the pipet. Such an external port may need to close when the pipette is pressurized to distribute its contents. The imaging array may be moved along various coordinate systems including non-limiting examples of cartesian, polar, cylindrical, spherical, and other such coordinate systems. By moving the imaging elements in space, deposits may be created in three dimensions.

### Methods of Producing and Utilizing Imaging Systems

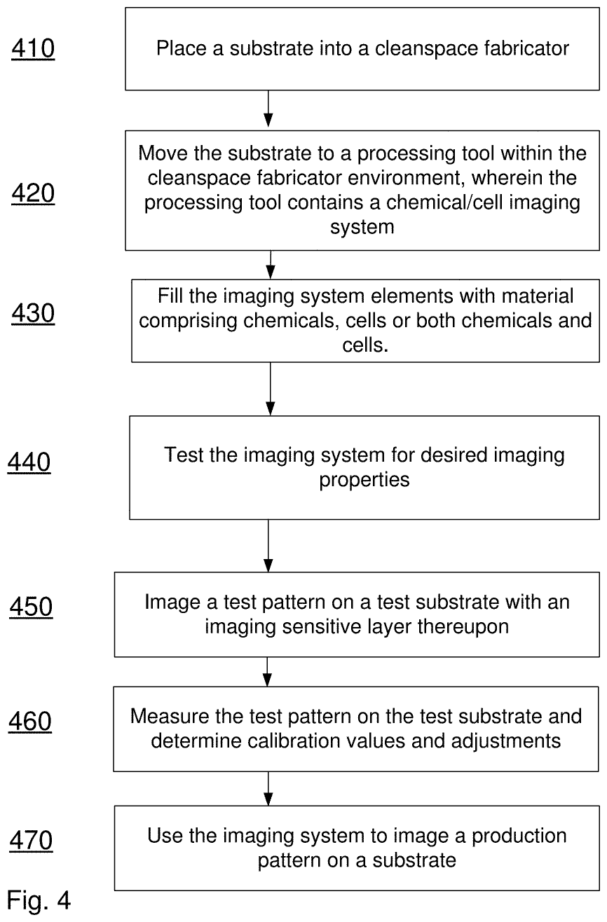

Referring to FIG. 4, a method for producing an imaging system may be found. At Step 410, a substrate may be placed within a cleanspace fabricator. At step 420 the substrate may be moved to a processing tool. In some embodiments, the processing tool may be located within a toolPod. At step 430 a processing step may be performed within the processing tool as part of a processing flow to form an imaging system. At step 440, the imaging components upon the substrate may be tested for their desired imaging properties. At step 450, the imaging system may be used to image a test pattern on a substrate with an imaging sensitive layer thereupon. At step 460, a metrology process may be performed on the substrate with the test pattern and calibration adjustments may be determined. At step 470 the imaging system may be used to image a production pattern on a substrate with an imaging sensitive layer thereupon.

### Control Systems

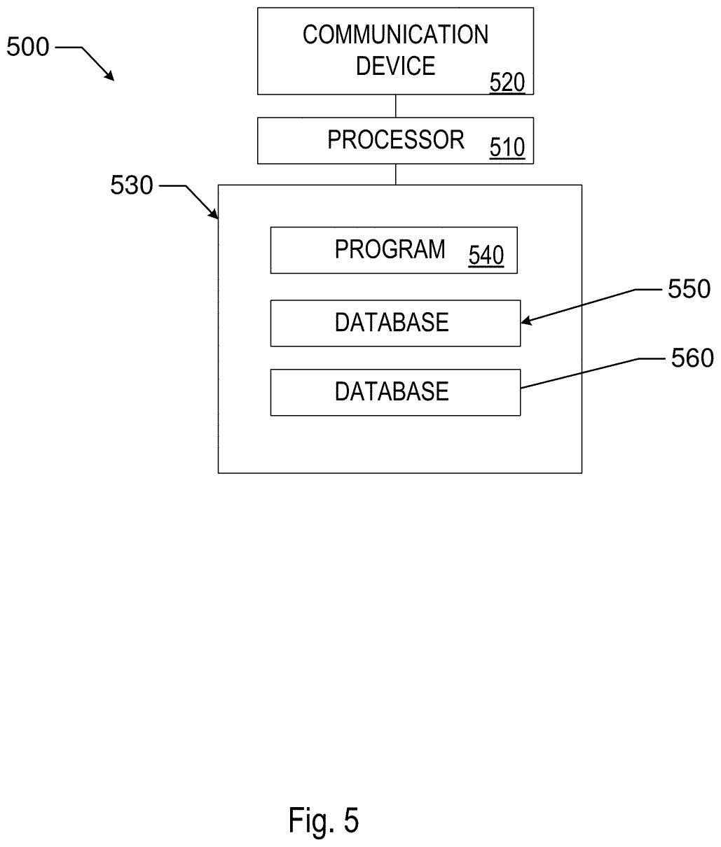

Referring now to FIG. 5, a controller 500 is illustrated that may be used in some embodiments of an imaging system. The controller 500 includes a processor 510, which may include one or more processor components. The processor may be coupled to a communication device 520.

The processor 510 may also be in communication with a storage device 530. The storage device 530 may comprise a number of appropriate information storage device types, including combinations of magnetic storage devices including hard disk drives, optical storage devices, and/or semiconductor memory devices such as Flash memory devices, Random Access Memory (RAM) devices and Read Only Memory (ROM) devices.

At 530, the storage device 530 may store a program 540 which may be useful for controlling the processor 510. The processor 510 performs instructions of the program 540 which may affect numerous algorithmic processes and thereby operates in accordance with imaging system manufacturing equipment. The storage device 530 can also store imaging system related data, including in a non-limiting sense imaging system calibration data and image data to be imaged with the imaging system. The data may be stored in one or more databases 550, 560. The databases 550, 560 may include specific control logic for controlling the imaging elements which may be organized in matrices, arrays, or other collections to form a portion of an imaging manufacturing system.

### Cell Printing

In some examples, the multiple print head devices as have been described may be used to print single cells upon a substrate. in some examples, a droplet containing a cell in a liquid media, such as growth media, may be printed. In some other examples, the cell may be printed alone. There may be numerous types of cells that may be printed at different locations determined by a model used to control the print head. The different cells may be grown from stem cell parents obtained or created from cellular material of a patient. Through various means, the stem cells may be differentiated and grown up to larger volumes of cells for printing. The multiple print heads may be fed in channels that form a row of print heads. In other examples, each print head may be positioned with its own reservoir that may contain a sample of cells for that print head alone. The print heads may be fed by reservoirs and piping and pipetting systems, or in some examples the print head may be married to a microfluidic processing element that may allow material to be distributed to any of the means of distribution to the print heads.

### Stem Cells and Biochemical Processing for Differentiation

In some examples, a large print head with many individual printing element, such as over 10,000 for example, may be used to print relatively large areas with cells of different types to form tissues with the deposition. In a non-limiting example, cells to be printed may be cells of an individual patient, where the printed cells are grown from a cell line that originates with the patient him/herself.

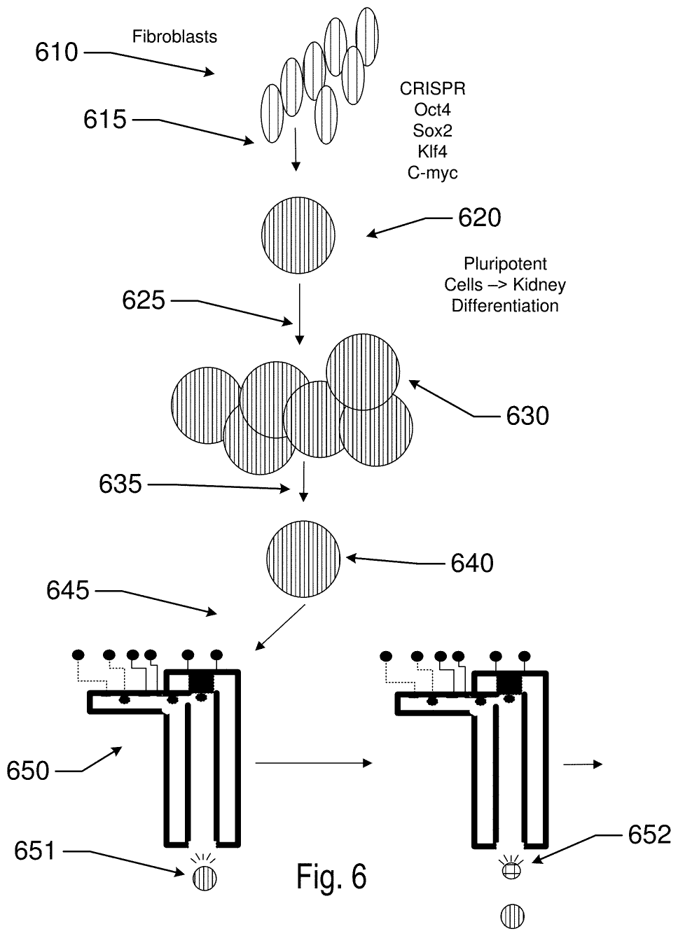

Referring to FIG. 6, an example of printing cells from a patient is illustrated. A sample of cells may be obtained from the patient such as the exemplary fibroblast cells 610 which may be isolated from a sample of a patient's skin. There may be numerous manners to induce the sample cells to become stem cells which will have the potential to grow and multiply. In a non-limiting example, genetic modification of the fibroblast cells may be performed. In an example, a transcription technique or gene editing technique 615 such as those based on CRISPR-Cas9 may be used to induce alteration of a series of genes such as the OCT4, SOX2, KLF4 and C-MYC genes which have been shown to induce pluripotency. The pluripotent cells 620 may be grown up and multiplied 625 to a collection of pluripotent kidney cells 630. In some examples, the growing collection of cells may be dissociated by physical or chemical means and separated 635. In some examples, separation of any cells that are not pluripotent may be accorded by the binding of antibodies to the cells that differentiate the different cell types. The different cells some with bound antibodies which may have a fluorescent marker attached or may be a substrate for an additional antibody that has a fluorescent marker may be sorted based on the fluorescent signals of the antibodies or other dyes. The separated individual pluripotent cells 640 may be loaded or passed 645 into the printing system. A printing system of the type herein may print 650 the cell 651 either in a droplet of media or by itself at a location that is determined by an algorithm that processes a model of the location of various cell types. In some examples, another material may be printed after the cell is printed. This additional material may include the addition of recombinant growth factors or small agonists 652 that may guide the pluripotent stem cell to differentiate into a desired type of cell for the location.

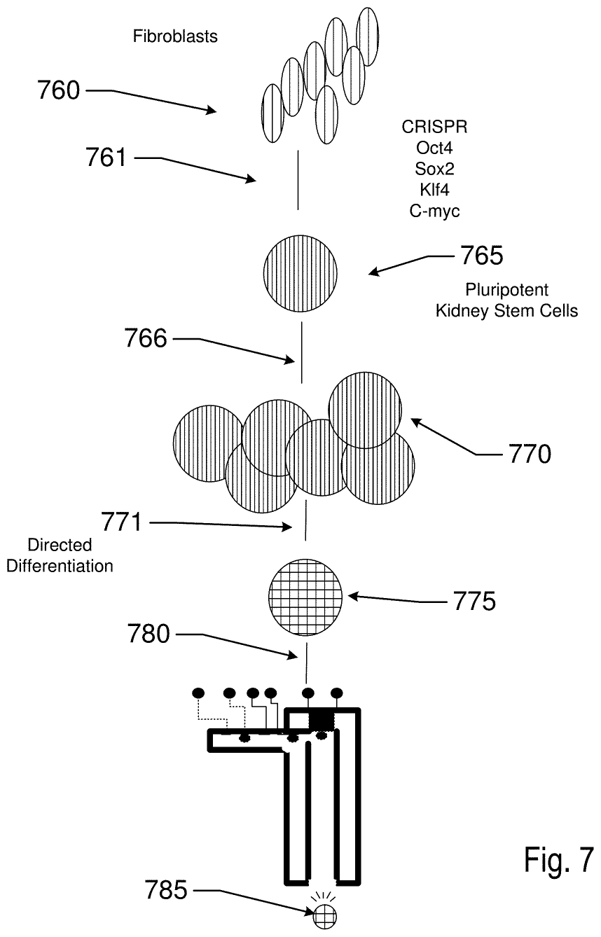

Referring to FIG. 7, a different printing scheme may be observed. A sample of cells may be obtained from the patient such as the exemplary fibroblast cells 760 which may be isolated from a sample of a patient's skin. There may be numerous manners to induce the sample cells to become stem cells which will have the potential to grow and multiply. In a non-limiting example, genetic modification of the fibroblast cells may be performed. In an example, a transcription technique or gene editing technique 761 such as those based on CRISPR-Cas9 may be used to induce alteration of a series of genes such as the OCT4, SOX2, KLF4 and C-MYC genes which have been shown to induce pluripotency. The pluripotent cells 765 may be grown up 766 to a population 770 and then influenced with the addition of recombinant growth factors or small agonists 771 to differentiate into various Kidney cell types 775. In some examples, the Kidney type differentiated cells can form embryonic forms of key Kidney elements including the nephron and early stage elements including the glomerulus and the uterine system. In some examples, the growing elements may be dissociated by physical or chemical means and separated. In some examples, separation may be accorded by the binding of antibodies to the cells that differentiate the different cell types and may be sorted based on the fluorescent signals of the antibodies or other dyes. Other separation schemes may be employed. The separated individual cell types 775 may be loaded or passed 780 into the printing system. A printing system of the type herein may print 785 the cell either in a droplet of media or by itself at a location that is determined by an algorithm that processes a model of the location of various cell types. In some examples, a collection of cells may be formed into a droplet or “ink” for printing. In some examples, another material may be printed after the cell is printed.

Other organ types or tissue types may be processed in analogous means. The examples relating to kidney cells are just one of many examples which may include skin, bone, heart, liver, colon, thyroid, brain, muscle, and other types.

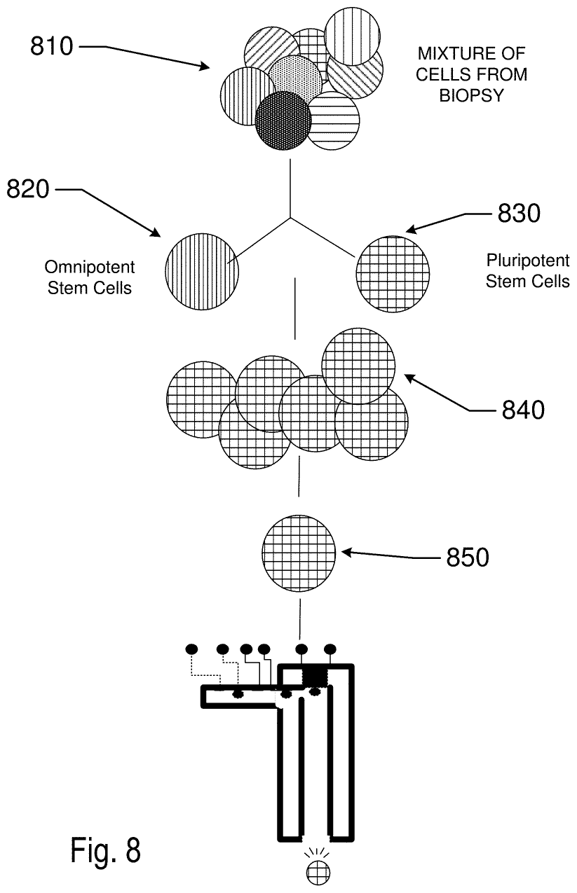

Referring to FIG. 8, an alternative method of printing cells is illustrated. A mixture of cells may be collected from a biopsy 810 of a patient. In some examples, the biopsy may include a small number of stem type cells. In some examples, which may be very rare, omnipotent stem cells 820 may be found. Such cells could be used for printing schemes. In other examples, pluripotent stem cells may be located within portions of an associated organ, such as kidney pluripotent stem cells 830. These pluripotent stem cells 830 may be grown up and multiplied 840. In some examples, the growing collection of cells may be dissociated by physical or chemical means and separated. In some examples, separation of any cells that are not pluripotent may be accorded by the binding of antibodies to the cells that differentiate the different cell types. The different cells some with bound antibodies which may have a fluorescent marker attached or may be a substrate for an additional antibody that has a fluorescent marker may be sorted based on the fluorescent signals of the antibodies or other dyes. The separated individual pluripotent cells 850 may be loaded or passed into the printing system. A printing system of the type herein may print the cell either in a droplet of media or by itself at a location that is determined by an algorithm that processes a model of the location of various cell types. In some examples, another material may be printed after the cell is printed. This additional material may include the addition of recombinant growth factors or small agonists that may guide the pluripotent stem cell to differentiate into a desired type of cell for the location.
Printing Tissue Films with Multiple Cell Types with Chemical Imaging System

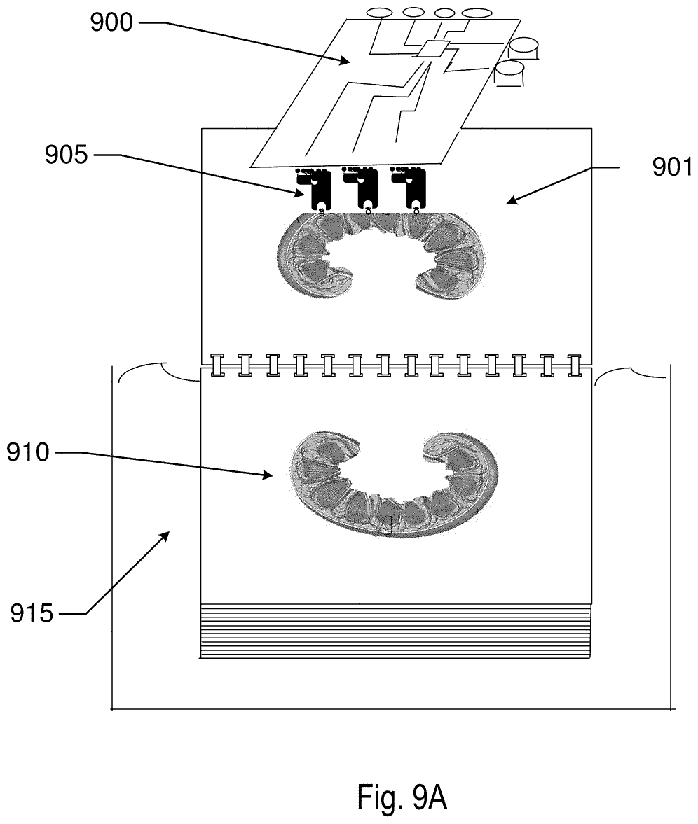

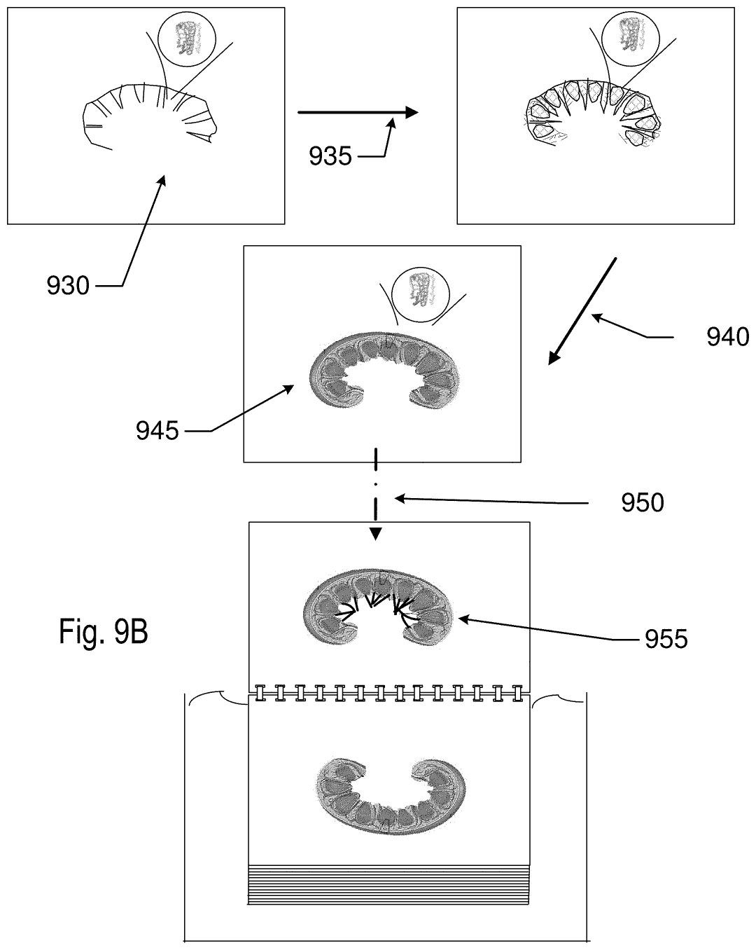

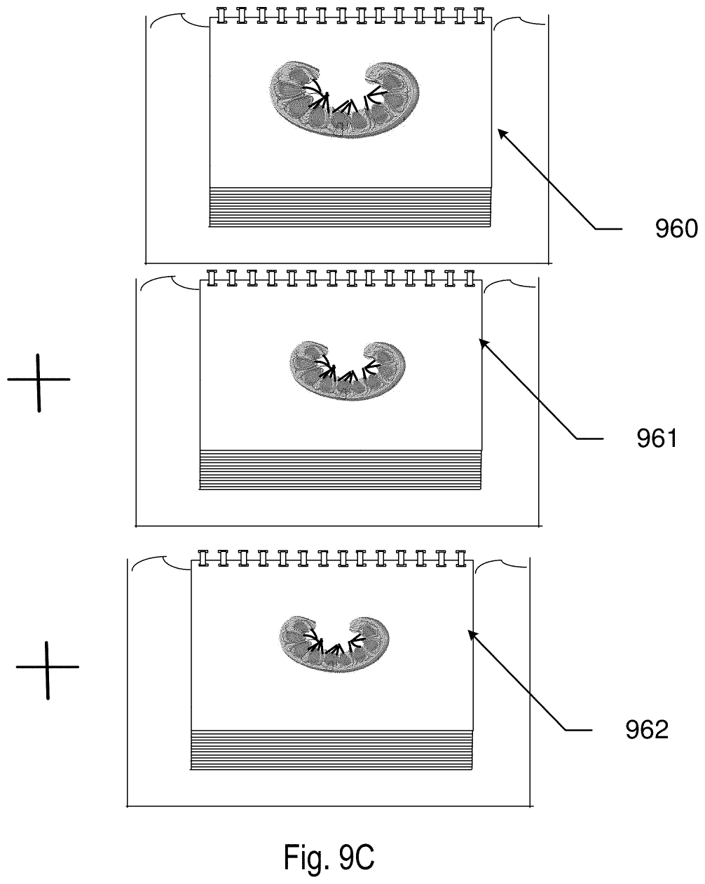

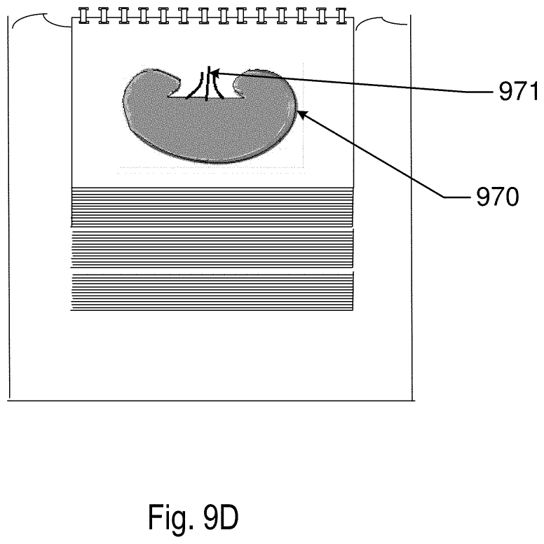

Referring to FIG. 9A, a method to print tissue layers using the concepts discussed herein is illustrated. A microfluidic processor with attached printing array element 900 is illustrated processing a flat substrate 901 to print 905 on tissue layers. The substrate may be formed of a variety of materials. In some examples, the substrate may be formed of biomaterials such as collagen or collagen related materials. In other examples resorbable materials from synthetic materials may be used. In some examples, the substrate may be processed to remove regions of the body of the sheet. Onto the substrate, cells may be printed resulting in a tissue layer 910 that may be stored in a nourishing medium 915. The cells may grow from the locations that they were printed in. Depending on the resolution of the printing system, small features may not be able to be imaged by the printing means.

Referring to FIG. 9B, another processing means such as techniques used in microelectronics processing may be used to form matrixes with small form factors. Techniques such as film deposition, resist deposition, reactive ion etching, chemical etching, and other such techniques may be used to form small structures 930. In an example of a kidney production, structure such as the nephron, glomerulus, uretic bud, and the like may have small structure used to create collections of cells that may grow into the small structures 935. Various means may be used to deposit cells of appropriate types upon the small support structures. The support structures may have molecules absorbed to them that attract certain types of cells to bind at appropriate regions. In other examples, layers of cells may be applied or printed in sequential processing to form small structures with differentiated cells in various locations. The sheets of material with the small structures may be applied 940 upon the other printed structures. A number of substrates with small structures 945 may be applied upon the previously printed tissue. In some examples, additional printing steps 950 may be used to print cells that may form vascular structure into appropriate regions of the growing layer which may inter-attach other formed structures 955. Collections of layers processed in the above manners, perhaps dozens or hundreds of such layers may be stacked upon each other and then allowed to grow. Referring to FIG. 9C there layers 960, 961 and 962 may be stacked upon each other. Referring to FIG. 9D, the multiple stacked layers 970 may grow into a formed organ. In some examples additional structures such as the renal veins and arteries 971 as well as ureter structures may be printed into locations between the layers.

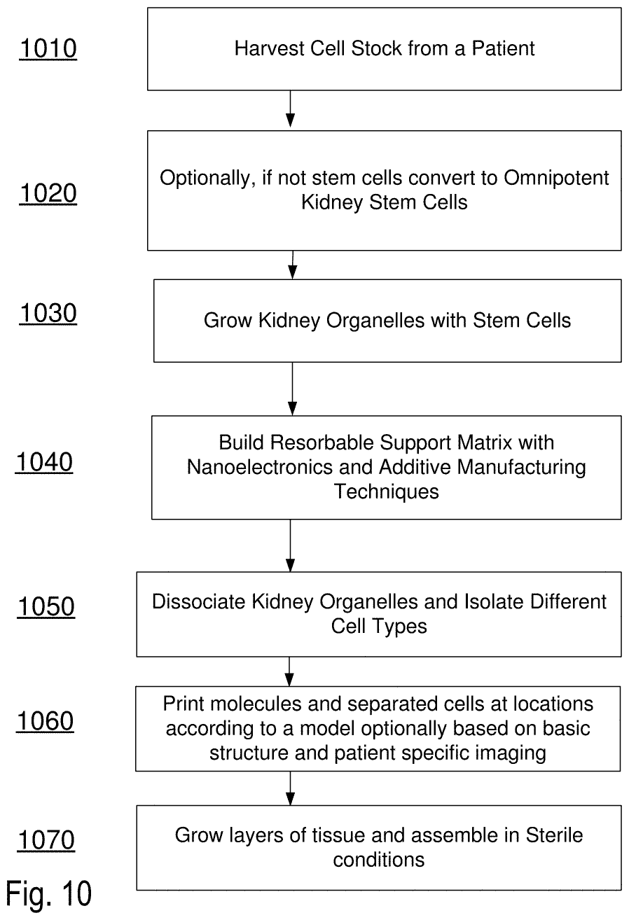

Referring to FIG. 10 an exemplary flow is illustrated. At Step 1010 a cell stock may be harvested from a patient. As mentioned earlier, the cell stock may be sorted to isolate existing stem cells from the patient including as a non-limiting example pluripotent stem cells from the Kidney. In other examples, other cells such as fibroblasts may be converted to omnipotent kidney stem cells at step 1020. The isolated or converted cells may be grown at step 1030 to form early stage growth or embryonic type growth of organ related components such as parts of the nephron, uretic body, venous system, and the like. In some examples, the growing organ components may be allowed to mature by placing them into a support matrix. In other examples, the early stage organ components may be separated into different cell types which may be further grown up and used to print structures with different cell types. At step 1040, a support matrix may be constructed to support printed cells or otherwise located cells. In some examples, the support matrix may be built to be resorbable into the growing organ tissue, such as from a collagen base for example. The support matrix may be constructed with various techniques include nanoelectronics techniques such as photolithography, reactive ion etching, chemical etching, and film deposition techniques as non-limiting examples. Additive manufacturing techniques may be used to place materials such as molecules of various types upon or into the support matrix. In some example, particular growth factors or other molecules that could support differentiated growth of cell types upon the support matrix may be added with additive manufacturing. In an illustrative example, a rod of support material may be used to lay out the structure of an artery or vein in a tissue layer to be formed. The rod may be printed with cells that surround the rod and grow into a venous form. The rod may include printings of growth factor to encourage or direct the growth of the appropriate differentiated cell type. Nanotechnology may be used to create small, controlled structures to form the support matrix.

As mentioned previously, at step 1050, grown structures of cells may be dissociated and then separated to form isolated collections of different cell types which may be fed to printing apparatus. At step 1060, the printing apparatus may be used to print both molecules and separated cells at locations according to a model formed to result in a desired organ or tissue layer. The model may be based on basic structural data and may be combined with patient specific imaging data. At step 1070, substrates formed as mentioned above may be placed in sterile locations with correct growth conditions to induce the growth of desired tissue layers. The layers may be assembled in the sterile conditions and allowed to further grow into more mature tissue layers. As the layers mature, fluids such as nutrient containing isotonic fluids may be flowed through the developing organ. The fluids may include blood simulants, or even blood of the patient at stages of the organ or tissue layer growth.

### Blood Contacting Devices

Numerous types of devices can be constructed to interact with a blood supply of a person or of a non-human animal. In some examples, allogeneic tissue and cell engineering products may be produced for use in patients. Due to differences in the surface expression of such cells, reactions may occur or be suppressed in the use of the product. In other examples, autologous sources of cells may be used to create products which may be less likely to be rejected or cause other interactions. There may be numerous tradeoffs between the two types including time scales involved to reach a needed number of cells to produce a product since stocks of allogeneic cells may be stored, such as in frozen form. Although the various products described in following sections may be processed using each type of cell initial stock, focus may be made on autologous processing for tissue and cell based applications and to other types of cell stock lines from various species types for vaccine and antibody production.

Devices formed of cellular based tissues may have numerous functions both in concert with an animal user and in some examples in use manners unconnected with an animal organism. In a class of examples used in concert with an animal, the animal's blood supply may be allowed to contact portions of the tissue engineering product. In some examples, such a product may be embedded in the user, in other examples it may be contained in a housing of some material and reside outside of the body of the user. The housing may be constructed of artificial materials in some examples, and in other examples may also be formed of tissues such as layers of endothelial tissues for example. In examples where the device resides outside of the user it may interact with the user's blood through an implanted blood access port. In other examples, connect may be made via intradermal means such as with arrays of intradermal needles. These needles may interact with interstitial fluids of the user which may indirectly interact with the blood system. In some examples, the structures such as ports and needles may be formed of user tissues or may be comprised of artificial materials.

Once a device has direct or indirect access to the blood system of a user it can be designed to perform various functions. In an example, a collections of tissues may be formed which perform the function of separating and ultimately removing materials from the user's system. In an example, an extra-corporal device may be comprised of cell layers from kidney related pluripotent stem cells and may form structures common with a kidney. In other examples, layers of cells may be assembled in a directed manner by printing processes that do not relate to natural growth patterns.

In an example, a tissue device may be created that has two dimensional or three dimensional layers that have an active or passive ability to move specific molecules, such as in a non-limiting example, triglycerides across tissue layers. In some examples, the movement may allow for separation of the molecules from the user fluids. In other examples, the movement may locate the molecules in regions of tissues where the molecules are metabolized.

In an example, a layered structure of tissue may include structures that allow both triglycerides and glucose to pass thru the tissue layer. On the other side of the tissue may be layers of muscle cells that are driven by an electrical signal to perform work. The muscle cells may metabolize the glucose. Other cellular layers may utilize the triglycerides.

In another class of examples, the separation of the glucose and triglycerides may move the molecules to a fuel cell location. The fuel cells may produce electrical energy from the molecules separated from the blood or other fluids of the users. In an example, such a device may allow a user to connect a fuel cell to his body and produce usable electricity. In some examples, such a device may function as a caloric drain on a user's body to facilitate weight loss.

In another example, a layer of capillary tissue may be grown to facilitate diffusion of glucose across the tissue layer into an adjacent space. The adjacent space may include a loosely dispersed layer of cultured adipocytes from the user's cells. A collection of extracorporeally located adipocytes may work to supplement activity of a user's body to respond to insulin signals in the blood stream and to segment glucose out of the blood stream either due to better performance of the adipocytes or by the increase in their number in connection with the blood stream or indirectly in connection with the blood stream through the production of other signaling related molecules.

In another example, a patient's cells may be used to grow adipocytes in volume. In some examples, adipocytes may be used to treat glucose related diseases such as diabetes. In some examples, layers of tissues including adipocytes and vascular tissue may be formed into a structure which may be connected to the circulatory system of a patient. Young adipocyte cells may be able to perform various bodily functions in manners superior to existing cells and treatment by flowing blood through the device may aid the patient. In other examples, implants may be created that man be placed into a patient's body for a similar function.

In another example, a layer of tissue may be configured to perform an action akin to kidney action, separating waste materials from the blood stream. In some examples, cells grown in layers may form structures that may aid in the separation of waste materials from the blood stream.

In another example, a layer of hepatocytes from a user may be constructed on a high surface area three dimensional matrix where a patch comprising the cells and matrices of needles may be used to detoxify the blood of a user. In other examples, a blood port or vascular puncture may be used to pass blood over the formed layers.

In another example the permeable layer of capillary based tissue may allow for gases from a space exterior to the layer to diffuse into contact with the blood. In an example, tissues from animals such as fish that extract oxygen from water may be combined to allow for concentration of oxygen underwater.

### Neuron Related Systems

The techniques that form tissues as discussed herein may be used to create novel devices relating to neurons. In an example, a combination of neurons and electronics may be formed to create interfaces for connection to electronic devices. A combination of electronic photodetectors and nerve cells may be formed for a biophotonic device. In other examples, nerve cells may be formed near electronic sensing devices, where the result of a firing of a neuron may be detected and cause an action, such as in a non-limiting example the firing of a led emitting diode circuit, for another type of biophotonic device. A cleanspace fabricator may be well suited for creating both tissue engineering products and electronic products as well as products that combine electronics and cell and tissue engineering.

In some other examples, stand-alone devices may be created with tissue engineering. For example, a created neural network may be designed and implemented with neurons printed into three dimensional structures upon support material. The network may also include vascular structure to allow for blood or artificial blood to be circulated through the device. Such a device may be artificially designed, and a type of programming may be performed through the adjustment of aspects of the interconnections between cells. Various types of computational devices may be formed for a form of neural computing.

There may be numerous examples in nature of sensory systems with highly performing capabilities. Bioelectronic systems with the exemplary capability may be created in a tissue engineering fab. For example, a nasal sensory system of various canine species may have sensory cells that can be isolated and/or grown from stem cells with appropriate signaling queues. The sensory cells may be paired to cultured nerve cells and deployed on three dimensional support structures that allow the nerve cell to connect to electronic sensors for readout. The mechanisms for introducing gas samples from the environment may be one or more of fabricated material or grown structures. In a fabricated example, diaphragms may be used to move air samples across the sensory cell structures where binding of molecules to appropriate sensory cells may create a detected sensing event. As well, pumps, valves and/or diaphragms may move fluids such as blood, artificial blood, high dissolved oxygen content solvents such as perfluorocarbons or the like to provide the sensory cells with nutriments and required gasses for healthy survival. In a similar manner auditory sensing systems may use a combination of tissue engineered structures for sound detection in concert with nerve cells. In still further examples, visual sensors such as found in the retina may be formed into light detecting structures which may interface with electronics. In some examples, very large area sensors may be formed which may interface with electronics. Various other structures such as lenses may be biologically formed or may be built of electroactive artificial structures. In some examples, a sensing system may include neural structures resembling ganglia in animals to build devices that may process sensed information in various neural manners before nerve to electrical connections are made to electronically receive data.

In some examples, various feedback mechanisms may be engineered using combinations of biological and electronic components. In an example, chemical feedback controls may be formed. For example, a collection of glucose sensing cells may be grown and printed into structures which may be interfaced with neurons in a collection for form a neural processing component whose output may trigger insulin producing cells to release a level of insulin. The programming of the neural processing device may be created by the manner that the cells are deposited upon substrates. A resulting device may be able to be created from a cell stock of a user and be encapsulated, for example in hydrogels and then placed subcutaneously in a user to support the body in glucose regulation.

In some examples, similar regulatory feedback processes may be created where the output of a neural structure may interface with electronics and the feedback ultimately may control a physical structure attached to the user such as a device capable of releasing a chemical, medicament, pharmaceutical, nutraceutical or the like. In an example, a level of an electrolyte in a body may be sense by a physiological response of a cell and associated neuron response. The resulting electrical signal may interface with a chemical releasing device that can release into the body of the user an electrolyte supply.

In some other examples, sensing may be performed by molecules or biological structures that can sense the presence of an infection and/or foreign body in the body. The resulting detection of an infection may be processed by a neural processing device comprised of neurons which may induce the release of an antibiotic, an immune system cell type or component, or other such immune system moieties in the vicinity. The ability to create customized collections of neurons may allow for more direct problem solving devices to be created with living cells of standard types and/or with neural interfaces to electronics for bio-electromechanical device creation.

The ability to grow muscle cells of various types may allow for unique bioengineered devices to be created. In an example, muscle cells may be configured into non-standard collections to perform novel functions. For example, a pumping mechanism may be created by a peristaltic force on a sheet connected to muscle cells where the pressure of the force may be transformed into a vane pump type device with pneumatics. In other examples, an exemplary sensing device as described previously may be embedded in a hydrogel structure capable of being placed in a body. A muscle cell structure may be added to give the hydrogel device the ability to move, such as with a flagella type structure. Small devices of this type may be able to perform functions in various intracorporeal locations.

### Organ Systems

In the examples provided herein, examples have been given related to Kidney and Heart cells, tissues, and organs. These examples are only illustrative for the many types of tissue and organs that may be created using the principals disclosed herein. For example, skin tissues, cartilage, bone, lymphatic, and vascular tissues may be formed in similar manners using the techniques and apparatus disclosed herein. Furthermore, many organ systems may similarly be processed or tissue layers of them may be processed including but not limited to heart, liver, pancreas, lung, spleen, stomach, intestine, brain, esophagus, thyroid, gall bladder and tongue as non-limiting examples. As well, these tissues and organs may be produced and used in various types of organisms including but not limited to humans. Body elements that may comprise various tissue types such as ears, eyes, nose, skin with hair, and the like may also be processed in the type of apparatus described here. Therefore, the examples are not meant to limit to just one tissue or organ type.

### Exemplary Implementations of Fabs

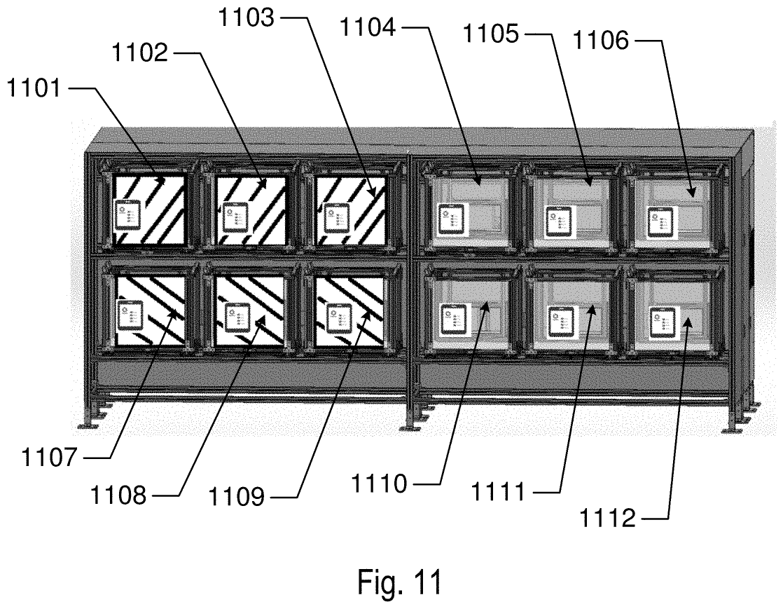

Referring to FIG. 11, an example of a tissue engineering fab according to various principles as have been discussed herein is illustrated. In a non-limiting sense, a collection of 12 toolPod positions is illustrated. The types of fabs may be completely scalable to larger and smaller collections of processing tools such as 1 to thousands of processing tools. A tissue engineering fab can derive significant benefit in the cleanspace fabricator design as the environment supports clean class environments as well as supporting genetic purity due to the lack of personnel in the fabricator bounds and supporting sterility since sterilization by various means may be accomplished in the cleanspace environments of the fab routinely, and perhaps even constantly.

At 1101, 1102 and 1103 a collection (illustrated with common hatching) of toolPods which have been interconnected is illustrated in concert. In some examples, the collection of 1102 and 1103 may contain processing tools related to cell culture. In some examples, the tooling combination may be dedicated to a single processing of a given cell type or cell genetic makeup. In other examples, samples of different cell types and genetic makeup may be introduced into the same tooling after cleaning cycles are performed. In the illustrated example, there may be multiple tools in the single toolPod 1102 such as multiple bioreactors from companies such as Eppendorf, PBS biotech, General Electric Healthcare, Pall, Solida and the like as well as bioreactor control systems such as that offered by Lab owl. The multiple tools may have their own encapsulations (which may cause them to be classified as toolPod subunits) where chemical tubing interconnects are used to make connection between the tools. The multiple tools may comprise different types of cell growing apparatus or may include a defined combination of different tools such as cell growth tools, cell counters, environmental control apparatus/adjustment devices and the like. In an example, the level of gasses such as CO2, oxygen, and water vapor as non-limiting examples may be controlled by apparatus both in growth media vessels as well as in the toolPod or toolPod subunit environments. Connections of the toolPods to various gas sources may be made through interfaces provided by the chassis to the toolPod, or they may be provided through a cable type connector with multiple utilities, gasses, electric and the like with an “umbilical” cord as a non-limiting example. In other examples a number of tools may reside in a single toolPod with interconnections between the tool residing in the same isolated space.

In some examples, the processing tools within the toolPod 1101 may include various analysis tools that can monitor and sense the performance of the cell culture processing steps. Examples in a non-limiting sense may include Fourier transform infrared spectrometers, confocal microscopy, ultraviolet spectroscopy, and the like.

The module may receive an initial cell stock in a number of manners. In some examples, the external portion of a toolPod such as 1103 may include a port through which a sample of cells may be introduced. In some examples, a needle may penetrate a membrane on the external face, in other examples a mechanized structure may pull a contained sample within the toolPod isolated space where it may be processed further to introduce the cell stock into the cell culture systems. In an example, toolPod 1104 may represent a dedicated material introduction system where various formats of cells may be introduced into the fabricator, and then the packaging sterilized as appropriate, and the contents identified and analyzed as appropriate before passing the material through a port and with the automation of the fab into other toolPods. In some examples, cells may be grown in or on microcarriers. One or more of the various tools may control the levels of dissolved oxygen in the growth media that the cells were confined in and/or these levels in the growth media may also be controlled by controlling the toolPod environments that surround these tools as well. Various means may be employed to control pH in the growth media Although specific current examples of tools that may be involved in cell growth/culture can be provided by examples in production today, the toolPod infrastructure allows for a flexible environment for many different processing tool types.

There may be many other factors that may be important for optimizing or enabling cell culture and growth. These conditions and factors may be adjusted and controlled by components of equipment in toolPods, the toolPods themselves or by components or materials containers that are attached onto toolPods, or by components of the fabricator facility that are operated to control select factors and conditions. In some examples, factors for control may include control of humidity, temperature, gas levels and other similar factors in the various fabricator, toolPods and equipment spaces.

In some examples, growth may occur in media of various kinds, the media may include various important components such as antibiotics, pH buffers, salts, and nutrients important in determining isotonicity and other critical parameters. Organic molecules such as growth factors, other proteins and the like may be added. In some examples, indicators of various types may be included to monitor and understand the control of growth conditions, spectrometric measurements of the conditions based on colorimetric changes in indicators may be used for automated control measurements in the various equipment and environments. In some examples, components of the growth media may be adjusted. In other examples, growth media may be changed or otherwise purified. Various flow control techniques may allow for isolating cell structures while smaller molecules and liquids are replaced. It may be important to remove growth media waste from the environment of the fabricator. In some examples, waste may be disposed of through waste facilities of the fabricator or through waste packaging made within toolPods or filled into containers temporarily attached to toolPods.

The toolPods may include interfaces on the external sides of the casing that may allow various forms of packaged materials to be held on the outside rear of the toolPod as it is involved in process. In some examples, materials such as growth media, supplies of gasses, and waste drainage may be held in bags, boxes, or other structures. In numerous examples single use formats formed from various polymeric materials may be interfaced with the toolPod and ultimately with the equipment within the toolPod.

In some examples one or more of the toolPods 1101-1103 and the like may include cell washing and harvesting equipment. The equipment may be used to replace growth media or to prepare samples of cultured cells for use in downstream processing such as bioprinting, plating and other uses of cells. Harvesting may involve numerous types of processing techniques including in a non-limiting sense centrifugal separation, acoustic based separation, counterflow centrifugation, and gravity flow based separation processors.

Concentrated and separated cellular product may be used in numerous downstream processing. The liquid containing the cellular product may be contained in numerous vessels and other types of substrates including microwell plates and the like. A substrate vessel, such as a plastic container may be sealed with a thermo-sealed or otherwise sealed lid in preparation for movement to other processing stations in a fabricator. In other examples, the collection of produced cells may be moved in a container that is not sealed but maintained in a clean and sterile environment of the fab. A covered or sealed substrate vessel may be moved within a primary cleanspace through a tool port and into a different tool port for further processing. In some examples, the product cells may also be transferred to a next toolPod for downstream processing. In some examples, the product cells may be contained in a three dimensional printing fluidic or microfluidic processing tool. The entire microfluidic processing tool may also be transferred through the fabricator primary cleanspace with substrate vessels containing the cell product or the substrate vessels may be moved along with a microfluidic processor to a printing station

The combination of toolPods 1101-1103 may have an exact copy 1107-1109 of the equipment deployed for cell culture. The exact copy may be used to culture a different source of cell stock. In other examples, the exact same sell stock may be grown in the second copy of equipment to minimize the risks involved during the growth process. In other examples, a different set of cell culture systems may be in toolPods 1105-1106 and separately in toolPod 1110.

In some examples, the toolPod 1108 may include analysis tools that may be able to probe and quantify aspects of grown cell stock as well as assembled tissues.

In some examples toolPod 1111 may be configured to perform tissue assembly and maturation. Tissue assembly may involve processing to plate out cell samples onto substrates without any patterning or imaging of the cell locations. In some examples, the tissue assembly equipment may include bioprinters of various types where the cultured and concentrated cell stocks from the previous processing toolPods may be patterned upon a substrate and patterned in a three dimensional pattern. In some examples, the patterning processes involved in tissue assembly may involve the creation or imaging of a scaffold in two dimensions or in three dimensions to support cells to grow in a pattern. In some examples, two dimensional assemblies of cells of various kinds may be processed with three dimensional printing to form a stack of layers that is incubated and allow to grow in a controlled fashion together.

In some examples, a toolPod may comprise an organ product and the fab automation is used to bring processing tools through the fab to the organ. In a non-limiting example, a collection of cultured cells may be assembled into a one-time use fluidic processing device that includes printing heads as part of its structure. The printing and fluidic device may be formed in toolPod 1112 as an example and then moved by the automation of the system to toolPod 1111 through a tool port. Once inside the toolPod 1111, the device may be received by the processing tool and calibrated in terms of its location. The printing device may be used to print one or more types of cells upon a growing organ structure, or in some examples upon a two-dimensional layer that is being stacked to form an organ. In some examples, the two-dimensional layer may have a film of bioabsorbable material upon which cells may be printed. In some examples, the layer of bioabsorbable material may be a mesh of material with holes, which may be smaller than a typical cell size, between fibers of the mesh. The printing unit that is passed through the tool port may include cell stocks that are cultured in other portions of the fabricator, and it may include various chemical mixtures that may be able to treat the surface of the bioabsorbable material in ways including providing local gradients of various nutriments, antibiotics, protein signaling molecules and the like to encourage or support the growth of different types of cells in a single incubated growth environment.

In an example, a toolPod 1111 may be loaded into a fabricator where the toolPod 1111 contains a previously processed substrate. In some examples the substrate may have a three dimensional model of support material that when filled or printed with cells can be organized to form an organ. In some examples, cadaverous organs of humans or animals which may have been reduced to their extracellular matrix may comprise the substrate for further cell printing or treatment. In other examples, an extracellular matrix analogue derived from imaging data or a priori model data generation may be used.

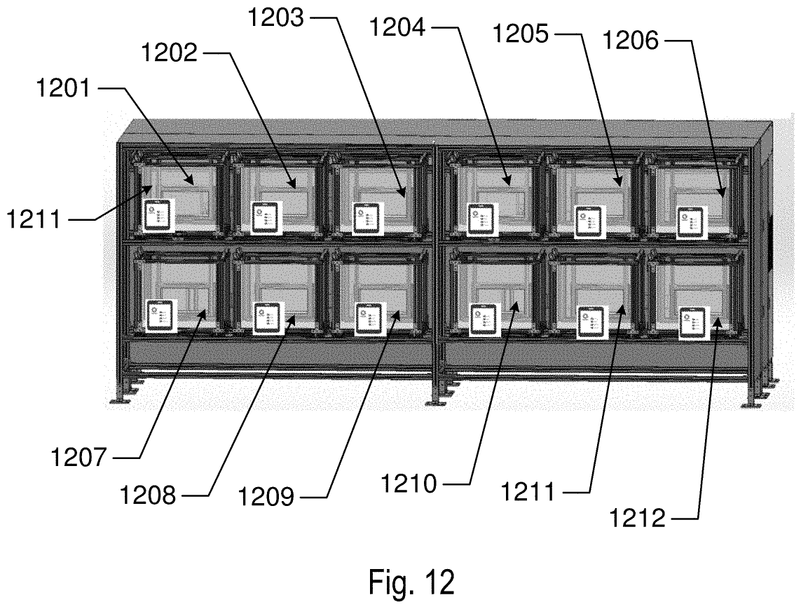

Referring to FIG. 12, an example of a vaccine or antibody production fab according to various principles as have been discussed herein is illustrated. In a non-limiting sense, a collection of 12 toolPod positions is illustrated. The tools of vaccine production may have analogous functions in production of antibodies or other biological products. The types of fabs may be completely scalable to larger and smaller collections of processing tools such as 1 to thousands of processing tools. A vaccine fab can derive significant benefit in the cleanspace fabricator design as the environment supports clean class environments as well as supporting genetic purity due to the lack of personnel in the fabricator bounds and supporting sterility since sterilization by various means may be accomplished in the cleanspace environments of the fab routinely, and perhaps even constantly.

In the following paragraphs, examples of vaccine and antibody production related to products related to severe acute respiratory syndrome coronavirus 2 (SARS-CoV-2) the virus that causes COVID-19 are included. But this contemporary example is offered in completely non-limiting senses for the examples. Other pathogens or targets for vaccines or antibodies may form equivalent examples of the application of the apparatus and methods discussed in the present specification and in referenced materials. Alternatives both for SARS-CoV-2 as well as other examples may also relate to the manners of implementing and using the apparatus and methods as disclosed.

In some examples, an exemplary vaccine or antibody fabricator may have materials and apparatus introduced into the operational environment. There may be numerous manners for this to happen. In some example, the fabricator may have material distribution aspects to provide gasses, liquids, and other materials to tools through defined interconnections. In other examples, materials may be introduced into tools through interaction with the toolPods from the periphery of the fabricator. In many examples, materials may be introduced into the internal spaces of the fabricator through defined input output equipment of the fabricator as a whole. At 1201 an exemplary input/output processing tool is illustrated. A portal of toolPod 1211 or door may allow for access to place a material or an apparatus inside the input/output processing tool. In some examples, the input/output processing tool may include numerous functions. In an examples, a means of disinfecting and cleaning a material or apparatus placed inside may be provided. In some examples, the entire toolPod and its contents may be raised to sterilizing temperature in another example, the materials may be subject to UV radiation, chemical sterilization, or a combination of sterilization techniques.

Other functions of the input/output processing tool may be to perform scans of the material or devices that are passed through the port. In some examples, entering material may be scanned for sizing, weight, or other physical characteristics. Materials contained in wrappings or containers may have labeling upon them which may be scanned optically for OCR, barcode or other information containing codes. RFIDs may be scanned. For materials leaving the fabricator, a bagging or containing capability may be performed and labelling of the packaging may be performed. In some examples, material characterization of various kinds depending on a product requirement may be performed, a non-limiting example of which may be a characterization for microbial content or lack of it. In other examples, the input/output may function just as a manner of controlling pass through of materials and attainment of sterility, whereas scanning and other characterization may occur in other toolPods.

In an example, a vial of a growth media may be placed in an input/output toolPod, the external surfaces may be sterilized by UV exposure within the toolPod, or the entire surfaces and contents may be thermally sterilized as appropriate for the material. In some examples, surfaces may be sterilized with “Steam in place” or “Clean in place” protocols as may commonly be used in cGMP operations. A robotic automation of the fab may then capture the vial and move it to another toolPod. In some examples, a glove handling capability may be provided to allow a user to place the vial upon automation of the fab. Automation controllers of the fab may interact with specialized controllers of the input/output toolPod to record, track and control data, images, scans, ID identification, environmental measurements, and the like. As well, tracking of materials according to good manufacturing practice (GMP) or other required protocols, procedures, registrations, and the like may be performed in automated fashions through the use of the input/output toolPod or toolPods and the handling of automation of the fab.

As has been described herein, there may be combinations of toolPods that have interconnection between them, both physically and through electrical and chemical tubing/conduits. These features are not illustrated for the exemplary vaccine and antibody fabricator, but they may be incorporated in manners as have been described.

In some examples, tools and equipment to perform DNA/RNA processing 1202, 1203 may be incorporated into one or more toolPods. For example, equipment to perform PCR protocols of various types such as, in a non-limiting sense, Amplified fragment length polymorphism (AFLP) PCR, Allele-specific PCR, Alu PCR, Assembly PCR, Asymmetric PCR, COLD PCR, Colony PCR, Conventional PCR, Digital PCR (dPCR), Fast cycling PCR, High Fidelity PCR, High-Resolution Melt (HRM) PCR, Hot start PCR, In-situ PCR, Intersequence specific (ISS) PCR, Inverse PCR, LATE (Linear-After-The-Exponential) PCR, Ligation mediated PCR, Long-Range PCR, Methylation-specific PCR (MSP), Miniprimer PCR, Multiplex PCR, Nanoparticle-Assisted PCR (nanoPCR), Nested PCR, Overlap extension PCR (OE-PCR), Real-Time PCR (Quantitative PCR (qPCR)), Repetitive sequence-based PCR, Reverse Transcriptase PCR (RT-PCR), Reverse-Transcriptase Real-Time PCR (RT-qPCR), RNAse H-dependent PCR, Single Specific Primer PCR, Single Specific Primer-PCR (SSP-PCR), Solid Phase PCR, Thermal asymmetric interlaced PCR (TAIL-PCR), Touch down PCR, Variable Number of Tandem Repeats (VNTR) PCR or other examples of PCR.

In some examples, the equipment in these type of “DNA/RNA” processing toolPods may be used for nucleic acid synthesis. Segments of DNA or RNA may be digitally designed or derived from sequencing experiments and then produced without intact cells i.e., “in vitro”, although “in vitro” processing may utilize cell derived materials, such as in a non-limiting example RNA polymerase. In some examples, a vaccine product may involve the creation of DNA plasmids that contain desired synthesized portions of DNA incorporated into an existing plasmid template. The initial processing may occur in these type of tools.

Some of the “DNA/RNA/processing tools may be used for synthetic/digital programming of DNA or RNA sequences for use in processes, in other examples the same or alternative tools may be used to measure, monitor and control processes in the fab by testing of samples, still further examples may involve the DNA/RNA tools being used to perform analytical tests on samples introduced to the fab, where an investigation of a genome of a particular pathogen in a sample may be of interest.

In some examples, a material containing plasmids or other DNA or RNA molecules that may have been synthesized or purified elsewhere may be introduced into the fabricator for further processing. In some examples, a sample of the externally submitted material may be studied by one or more of the techniques mentioned. There may be numerous types of purification and isolating kits and equipment that may function in a toolPod of these types.

In some examples, growth of cells in bioreactors or in vitro RNA synthesis in reactors may occur in exemplary reactor toolPods 1204, 1205, and 1206. The bioreactors may be used to grow various types of cells in well controlled conditions. For example, some types of vaccine products may be grown in standard cell lines. Examples may include influenza vaccines produced in insect cells, or in mammalian cells such as MDCK, CHO or other such standard cell lines which may also be adapted for various processing enhancements for particular processing. Rotavirus vaccines may also similarly be produced in mammalian cell growth environments for bioreactors. Measles, smallpox, Polio, Rabies, and Japanese Encephalitis may all be other examples of vaccines produced in a primary cell line grown in a bioreactor. In some of these examples, the cell lines produce copies of the virus, and further processing may weaken or inactivate the viruses to produce a vaccine product. In other examples, inactivation of the produced viruses may be desirable.

In other examples, a vaccine product to act against a primary virus target may be produced by growing cells in bioreactors where the cells produce abundant copies of a secondary and different virus type as a viral vector. The viral vector may have been genetically modified to comprise DNA or RNA, as appropriate, of the primary virus target. The DNA modifications may allow the viral vectors to express proteins relevant to the primary virus target. In an example of a SARS-CoV-2 vaccine product, a protein target of the primary SARS-CoV-2 virus may be one of its proteins, so-called “Spike” protein, a roughly 1000 amino acid protein believed to be used by the virus to bind to receptors such as the ACE2 receptor on certain human cells. In some examples the DNA coding for the spike protein may be introduced into specialized cells which will then express all the necessary components as well as the inserted DNA sequence or an associated RNA strand based on the inserted DNA sequence to create a replication incompetent virus vector.

These specialized modified cell lines can be grown in bioreactors such as 1204, 1205 and 1206 for example. In some examples, the modified cell lines may multiply in a bioreactor system without creating the viral vector product and then when a high amount of the cells have been produced, they may be induced by various manners to create the viral vector product. In a non-limiting example, the change in the production may be affected by introducing a particular sugar molecule such as arabinose. Therefore, the bioreactors 1204, 1205 and 1206 may include capabilities to sense growth conditions by various means such as by photometric means and then trigger flows of reactants into the growth reactors when a level of growth reaches a target amount. Other means of measuring growth may include light scattering techniques, sensing of various chemical signals relating to growth or depletion of components of the growth media and the like.

The growth may generate large amounts of the genetically modified virus vector. Thus, a vaccine with the exemplary virus vector that is grown in cells such as mammalian cells as a non-limiting example may be used to elicit an immune response in a host that would be protective against a primary virus target. In some cases, the virus vector may be engineered so that the resulting virus may itself be able enter host cells—as a “pseudo” infection, but it may not be capable of creating new functional virus particles. Because the relatively benign virus vector will make it possible for the pseudo-infected cells to generate large amounts of the SARS-CoV-2 protein, the host immune system can be trained to respond to SARS-CoV-2. A non-limiting list of examples of viral vectors that may be grown in the bioreactor tool pods to create either DNA or RNA vectors may include versions of adenovirus, vesicular stomatitis virus, and measles as well as others.

In some examples, a eukaryotic or prokaryotic production cell line may be created to express viral, bacterial, or in general microbial proteins in abundance as it grows. In some examples, the cell lines may be created to produce subunit vaccines, e.g., proteins that in some examples may be soluble or may self-assemble into products. And, for example, DNA plasmids may be produced by E. coli.

After growth in a reactor toolpod such as reactors 1204, 1205 and 1206 the cells may be lysed and then the resultant product may be purified to isolate the desired protein antigens. In some examples, specially formulated adjuvants, which may stabilize products and/or stimulate an immune response, including some that may bind the antigens may be used to formulate the vaccine. In a non-limiting example, an adjuvant based on nanoparticles with high surface area may bind the antigen to present highly concentrated antigen solutions.

In some examples, the reactors 1204, 1205 and 1206 may not function to grow up cell based products. Biological reactions may be performed “in vitro” in the reactors. As an example, a reaction media may be configured into a reactor 1204 containing DNA substrate, protein machinery and nucleotides and/or nucleic acids of various types important to the production. The reactor may be used to create protein based products, or DNA products such as plasmids, or RNA products such as messenger RNA strands engineered to produce desired protein products or other biological products in host cells. In the illustrated example, there may be multiple tools in the single toolPod 1204 such as multiple bioreactors from companies such as Eppendorf, PBS biotech, General Electric Healthcare, Pall, Solida, Univercells and the like as well as bioreactor control systems such as that offered by Lab Owl. The multiple tools may have their own encapsulations (which may cause them to be classified as toolPod subunits) where chemical tubing interconnects are used to make connection between the tools. The multiple tools may comprise different types of cell growing apparatus or may include a defined combination of different tools such as cell growth tools, cell counters, environmental control apparatus/adjustment devices and the like. In an example, the level of gasses such as CO2, oxygen, and water vapor as non-limiting examples may be controlled by apparatus both in growth media vessels as well as in the toolPod or toolPod subunit environments. Connections of the toolPods to various gas sources may be made through interfaces provided by the chassis to the toolPod, or they may be provided through a cable type connector with multiple utilities, gasses, electric and the like with an “umbilical” cord as a non-limiting example. In other examples a number of tools may reside in a single toolPod with interconnections between the tool residing in the same isolated space.

Many examples of producing vaccines and growing them in reactors within a toolpod are provided, however, very similar processing may be used to produce antibody products. For example, cloned cells which may be genetically programmed to produce effective antibodies may be grown in an exemplary bioreactor. For example, a rabbit cell based hybridoma formed by fusion with myeloma. Selective factors in the growth medium may be used to target the desired cells which will produce large quantity of antibody which may then be purified to derive product.

In some examples, the processing tools within the toolPod 1204 may include various analysis tools that can monitor and sense the performance of the cell culture processing steps. Examples in a non-limiting sense may include Fourier transform infrared spectrometers, confocal microscopy, ultraviolet spectroscopy, and the like.

The module may receive cell stocks, growth media in a number of manners. In some examples, the external portion of a toolPod such as 1204 may include a port through which a sample of cells may be introduced. In some examples, a needle may penetrate a membrane on the external face, in other examples a mechanized structure may pull a contained sample within the toolPod isolated space where it may be processed further to introduce the cell stock into the cell culture systems. In an example, input/output toolPod 1201 as discussed may be a dedicated material introduction system where various formats of cells may be introduced into the fabricator, and then the packaging sterilized as appropriate and the contents identified and analyzed as appropriate before passing the material through a port and with the automation of the fab into other toolPods including the bioreactor toolPods 1204, 1205 and 1206. In some examples, cells may be grown in or on microcarriers. One or more of the various tools may control the levels of dissolved oxygen in the growth media that the cells were confined in and/or these levels in the growth media may also be controlled by controlling the toolPod environments that surround these tools as well. Various means may be employed to control pH in the growth media. Although specific current examples of tools that may be involved in cell growth/culture can be provided by examples in production today, the toolPod infrastructure allows for a flexible environment for many different processing tool types.

There may be many other factors that may be important for optimizing or enabling cell culture and growth. These conditions and factors may be adjusted and controlled by components of equipment in toolPods, the toolPods themselves or by components or materials containers that are attached onto toolPods, or by components of the fabricator facility that are operated to control select factors and conditions. In some examples, factors for control may include control of humidity, temperature, gas levels and other similar factors in the various fabricator, toolPods and equipment spaces.

In many of the examples, a desired product of the reactor production may be mixed with a number of other materials. For example, the cells used to produce the product may lyse on their own as the production occurs, or they may be lysed intentionally. In some examples, toolPods 1207 and 1208 may contain processing equipment to purify the desired product from other components of the mix. There may be numerous techniques to perform the separation and purification. For example, types of chromatography may be performed. high pressure liquid chromatography may be used. Columns that separate the desired product may include affinity columns where the surface of the column filling materials may contain bound materials that may have affinity for the desired product so that as the product supernate is passed over the column, undesirable proteins, cellular components, and the like may pass through the column and be separated. In some examples, precipitation or flocculation techniques may be used to separate the desired products from undesired impurities. There may be multiple stages of purification where a bulk separation technique may be followed by a high purification step. Other methods for separation may include ultracentrifugation, tangential-flow filtration, and enzymatic digestion. Charged depth and membrane filters may be used to filter out impurities. Chemical methods may be used for bulk impurities reduction, coupled with more precise technologies such as chromatography may be employed.

In some examples, the purified product may be the product that proceeds to fill/finish processing. For example, isolated DNA fragments, proteins, and engineered virus particles may be finished products of the purification stage. In other examples, products such as messenger RNA may be packaged into liposomes or other micro/nanoscale containment.

Purified product may next be packaged for use or storage. In some examples, toolPods 1209, 1210, and 1211 may be used for fill finish processing. In some examples, preformed vials, syringes, and other storage items may be filled with the purified product. In other examples additional processing may occur to tailor the purified product with additional additives. In some examples, the storage items or syringe bodies may be formed in place and filled such as with blow fill seal technologies or form fill seal technologies. In some examples, three dimensional printing technologies, such as in a non-limiting example rapid SLA printing, may be used to print vials and syringe bodies which may then be immediately filled. In some examples, a liquid sample may be lyophilized to a concentrated liquid or to a powder form. The vaccine products may be better stored or processed under reduced temperatures, and the fill finish processing toolPods 1209, 1210 and 1211 may operate under reduced processing temperatures or under different ambient for product stability. In some examples finished product may be further processed to be surrounded in packaging to maintain a sterile environment around the filled products when they are removed from the fab. In some examples, the products may be removed through the toolPods directly. In other examples, the products may be transferred through the vaccine fab to an input/output toolPod 1201 or through a dedicated output toolPod 1212 in non-limiting examples.

### Modular Processing Systems

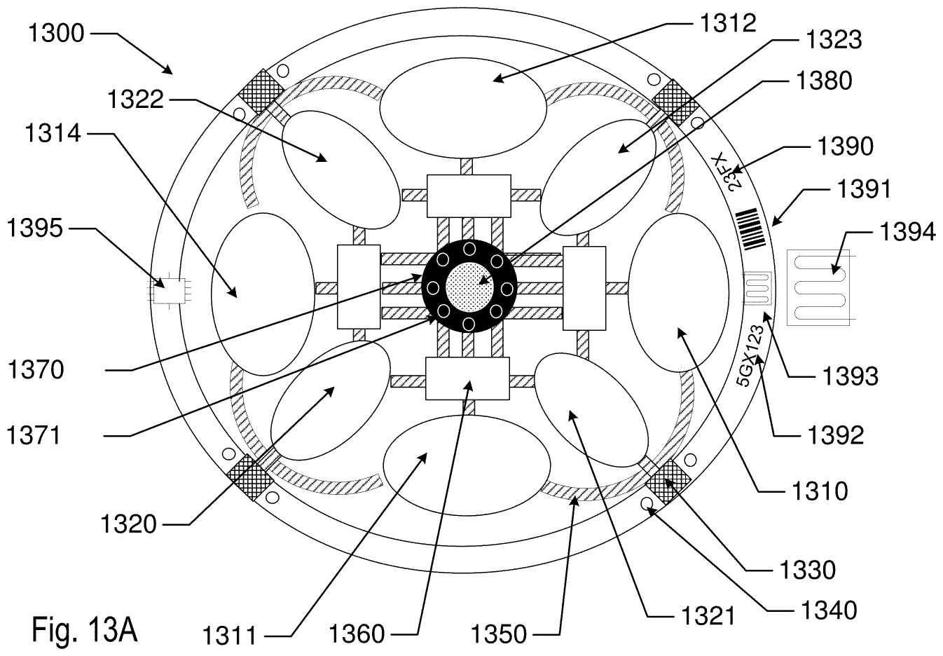

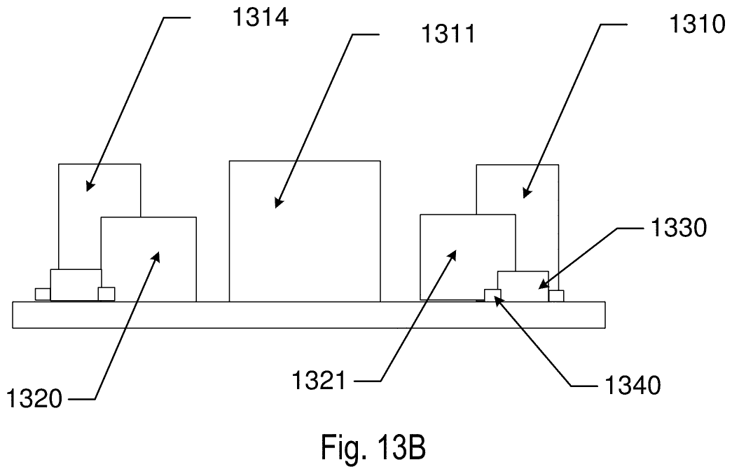

Referring now to FIG. 13A, a specialized processing and purification module for single use operations is illustrated. In some examples, the module may be designed for multiple uses and may be formed in similar manners with different materials such as stainless steel. Focus here will be on examples related to single use.

In the non-limiting example of FIG. 13A, a module 1300 or insert device that may be introduced into one or more of the exemplary processing tools of a fab of the types that have been described herein may function much like a toner cartridge in a laser printer. The module 1300, is an example of a module with integrated functions of growth and purification. A disk shaped module is illustrated as a non-limiting example. Such a shape may allow for the entire module to be used for centrifugation processing in a processing tool. The module 1300 may be formed of molded parts that are joined together. The module 1300 may have numerous important regions such as the various channels 1350. These channels 1350 may be formed a milli-fluidic or in some cases microfluidic type of processing features. The channels 1350 may be coated with various coatings and surfactants to give different characteristics that may be desirable for certain organisms that may be grown in the module 1300. Accordingly, different models and versions of a module may be made for different types of processing. For example, in some of the SARS-CoV-2 processing examples, a growth process with MDCK cells may be performed and these cells may prefer environments. Specialized forms may then be possible. In other examples, a standard device with standard surface treatment may be performed. In some examples a module may have growth vessels 1310,1311,1312, and 1314 as examples. These vessels may be connected to various interconnections such as tubes for gases such as oxygen, for exhaust, and for routing samples of materials for testing or sensing. In some examples, a module may be prefilled with growth medium for a particular application. In other examples, growth medium may be added either just before use or within the processing tool after the module has been placed into the processing tool.

The processing tool may engage or hold the module 1300 at an exemplary hub 1370 at the center of the module 1300. The hub 1370 may also have various interconnection devices 1371 that may allow the processing tool to create sealed interconnections with the module. The center of the hub 1370 may be a cutout 1380. Thus, a spindle of a processing tool may engage and center the module 1300 when it is inserted. In some examples, the module 1300 may have a coil of conductive material 1393 on its periphery. The coil of conductive material 1393 may be used to wireless conduct electrical energy into the module from an external coil 1394 for various purposes. The coils may be used to orient the module 1300 into a desired rotational orientation in space. In some examples identifying marks or RFID tags may be placed on the body of the module 1300 such as a text form serial number 1392, a bar code identifier 1391 which may also have an RFID under it, and a model number 1390 for the module 1300.

The processing tool may provide gasses, liquids, and the like at hub interconnects 1371 or at other interconnects 1340 which may be located at different positions on a module 1300. The processing tool may surround the module 1300 completely and may control temperature and the like either for the entire module 1300 or for select portions such as keeping growth modules 1310 and 1314 at 25 degrees Celsius, whereas keeping growth modules 1311 and 1312 at 37 degrees Celsius. In some examples, the processing tool may have sensing apparatus that may see into the body of the module 1300 at select points such as test point 1330. The module 1300 may also have sensing elements with these test points 1330 which may be capable of sensing conductivity, dissolved gases such as O2 and CO2 and the like. In some examples, microfluidic sensing elements which may include single use sensors may be used to measure various chemical and multi-omic signatures. In other examples, a sample of material may be removed from the module 1300 at a test port through interconnect 1340 as an example.

The module 1300, may included filtering and purification devices 1320,1321,1322, and 1323 of various kinds to perform chromatography, filtration, and other separations as appropriate. For modules with specific production goals affinity columns may be configured within a certain module type. High pressure liquid chromatography, centrifugation and other separation processes may also be performed. In some examples, purification may involve numerous types of processing techniques including in a non-limiting sense centrifugal separation, acoustic based separation, counterflow centrifugation, and gravity flow based separation processors.

The various sections of the module 1300 may have flow control components 1360 that may be interconnected to different vessels and to different purification devices. The fluid control components 1360 may include valves, pressure, and flow regulators. The valves may be on/off or flow restriction valves or may be valves that direct flow into selectable tubing paths. In some examples, the vessels may have fluid intermediates and products moved into to them from other regions of the module. Due to the relatively standard design of the modules, small sized vessels may be used to make batches of vaccine or antibody product. For a given output need, economies of scale and ease of production of the modules 1300 may result in improved economics. In some examples, a pharmaceutical grade plastic material such as polypropylene, polyethylene, polystyrene or coated versions as non-limiting examples may be molded, blow molded, or extruded into the basic shape of the module. Various microcarrier materials may be added to the growth vessels before the pieces are sealed together. In some examples, the purification devices 1321 may be added to the module 1300 before it is sealed together. In other examples, interconnects on the module may be sealed to purification devices 1321 at a later time, especially when higher operating pressures may require different material choices. Single use pumping elements may be included in the purification devices. Lower pressure and slower throughput chromatography solutions may be used with the smaller volume processing sizes. The flow components may also connect to external components through the hub 1370 and interconnects 1371.

Various sensor may be incorporated into the module as illustrated at one example position where test points 1330 may include the various sensing elements. In some examples, there may be electronics incorporated into the module to support the operations and the flow of data into and out of the module. An integrated circuit module 1395 may be located in the module and may have an integrated battery system, which in some examples may be charged inductively through a coil of conductive material 1393. It may be useful for the onboard electronics which may be connected to a number of sensing elements to be able to recognize patterns in the data coming from the sensing elements. In some examples, the data from the sensing elements may be analyzed with machine learning or artificial intelligence algorithms running on the integrated circuit module 1395. In some examples, an artificial intelligence chip may be incorporated into the module, sometimes on the integrated circuit module 1395, and it may be able to process data from sensors on the module, and data communicated from the processing tool interacting with the module 1300. The algorithms used in the artificial intelligence chip may be downloaded wirelessly or in a wired fashion to the module 1300.

Referring to FIG. 13B, the module 1300 is illustrated from a side view illustrating exemplary aspects of the relative height of the features shown on FIG. 13A. The growth vessels 1310, 1311, and 1314 being relatively higher than the exemplary purification devices 1320 and 1321. The test point 1330 is shown on the edge of the device, along with interconnects 1340.

Various sensing apparatus may be used to monitor the various environments in the fabricator. Some sensing apparatuses may be fab-wide and directly coordinate with controllers and data processors of the fab. Some sensing apparatuses may be associated with toolPods and coordinate with fab-wide systems directly or through communication systems of the toolPod. And, other sensing apparatus may operate at the equipment level, where the sensed data may be communicated from equipment directly to fab systems, directly to cloud/hosted control systems or to the toolPod first.

### Glossary of Selected Terms

Reference may have been made to different aspects of some preferred embodiments of the invention, examples of which are illustrated in the accompanying drawings. A Glossary of Selected Terms is included now at the end of this Detailed Description.

Air receiving wall: a boundary wall of a cleanspace that receives air flow from the cleanspace.
Air source wall: a boundary wall of a cleanspace that is a source of clean airflow into the cleanspace.
Automation: The techniques and equipment used to achieve automatic operation, control, or transportation within a cleanspace fabricator.
Clean: A state of being free from dirt, stain, or impurities—in most cases herein referring to the state of low airborne levels of particulate matter and gaseous forms of contamination.
Cleanspace (or equivalently Clean Space): A volume of air, separated by boundaries from ambient air spaces, that is clean.
Cleanspace, Primary: A cleanspace whose function, perhaps among other functions, is the transport of jobs between tools.
Cleanspace, Secondary: A cleanspace in which jobs are not transported but which exists for other functions, for example as where tool bodies may be located.
Cleanroom: A cleanspace where the boundaries are formed into the typical aspects of a room, with walls, a ceiling, and a floor.
Fab (or fabricator): An entity made up of tools, facilities and a cleanspace that is used to process substrates.
Periphery: With respect to a cleanspace, refers to a location that is on or near a boundary wall of such cleanspace. A tool located at the periphery of a primary cleanspace can have its body at any one of the following three positions relative to a boundary wall of the primary cleanspace: (i) all of the body can be located on the side of the boundary wall that is outside the primary cleanspace, (ii) the tool body can intersect the boundary wall or (iii) all of the tool body can be located on the side of the boundary wall that is inside the primary cleanspace. For all three of these positions, the tool's port is inside the primary cleanspace. For positions (i) or (iii), the tool body is adjacent to, or near, the boundary wall, with nearness being a term relative to the overall dimensions of the primary cleanspace.
Tool: A manufacturing entity designed to perform a processing step or multiple different processing steps. A tool can have the capability of interfacing with automation for handling jobs of substrates. A tool can also have single or multiple integrated chambers or processing regions. A tool can interface to facilities support as necessary and can incorporate the necessary systems for controlling its processes.
Tool Body: That portion of a tool other than the portion forming its port.
Tool Chassis (or Chassis): An entity of equipment whose prime function is to mate, connect and/or interact with a toolPod. The interaction may include the supply of various utilities to the toolPod, the communication of various types of signals, the provision of power sources. In some embodiments a Tool Chassis may support, mate, or interact with an intermediate piece of equipment such as a pumping system which may then mate, support, connect or interact with a toolPod. A prime function of a Tool Chassis may be to support easy removal and replacement of toolPods and/or intermediate equipment with toolPods.
toolPod (or tool Pod or Tool Pod or similar variants): A form of a tool wherein the tool exists within a container that may be easily handled. The toolPod may have both a Tool Body and also an attached Tool Port and the Tool Port may be attached outside the container or be contiguous to the tool container. The container may contain a small clean space region for the tool body and internal components of a tool Port. The toolPod may contain the necessary infrastructure to mate, connect and interact with a Tool Chassis. The toolPod may be easily transported for reversible removal from interaction with a primary clean space environment.
Tool Port: That portion of a tool forming a point of exit or entry for jobs to be processed by the tool. Thus, the port provides an interface to any job handling automation of the tool.
Vertically Deployed Cleanspace: a cleanspace whose major dimensions of span may fit into a plane or a bended plane whose normal has a component in a horizontal direction. A Vertically Deployed Cleanspace may have a cleanspace airflow with a major component in a horizontal direction. A Ballroom Cleanroom would typically not have the characteristics of a vertically deployed cleanspace.

The various examples of cellular and tissue engineering processing and vaccine and antibody product processing and constructs related to these may be developed and manufactured in the type of environment that has been described in these examples. However, the generality of cleanspace processing examples may be used for a multitude of different types of processes and the discussion of specific examples does not limit the generality to other processes. Likewise, the specific processing examples that have been discussed may have a preferred processing environment using the concepts as discussed here, however, they too may be carried out in numerous other types of environments such as laboratories and cleanroom facilities in some examples.

While the invention has been described in conjunction with specific embodiments, it is evident that many alternatives, modifications, and variations will be apparent to those skilled in the art in light of the foregoing description. Accordingly, this description is intended to embrace all such alternatives, modifications and variations as fall within its spirit and scope.

What is claimed is:

1. A biological processing apparatus, the biological processing apparatus comprising:
a cleanspace fabricator, wherein the cleanspace fabricator is configured to process at least a first substrate comprising biological materials, wherein the cleanspace fabricator maintains both a particulate cleanliness as well as a biological sterility cleanliness, wherein the cleanspace fabricator comprises at least a first processing apparatus and a second processing apparatus deployed along a periphery of the cleanspace fabricator, and wherein the cleanspace fabricator comprises fabricator automation to move one or more of the first substrate and the first processing apparatus within a primary cleanspace of the cleanspace fabricator;
a first toolpod and a second toolpod, wherein the first toolpod and second toolpod comprise at least a first fluid tubing that flows between the first toolpod and second toolpod;
a third toolpod comprising a bioreactor, wherein the third toolpod when placed within the cleanspace fabricator occupies a position of one of:
being above the first toolpod, or
beneath the first toolpod in vertical location;
wherein the first fluid tubing is connected between the first toolpod and the second toolpod with assistance of the fabricator automation; and
a fourth toolpod comprising an input/output station, wherein the input/output station comprises a sterilization device to sterilize a material placed into the input/output station, and wherein the fabricator moves the material placed into the input/output station from within the input/output station to within the primary cleanspace.

2. The biological processing apparatus of claim 1 further comprising
a fifth toolpod comprising a fill/finish processing equipment;
wherein the first toolpod comprises at least a first chromatography column; and
wherein the second toolpod comprises at least a second chromatography column.

3. The biological processing apparatus of claim 2 wherein the bioreactor comprises a genetically modified mammalian cell type, wherein a genetic modification of the genetically modified mammalian cell type encodes for a protein expressed on the surface of a microbe.

4. The biological processing apparatus of claim 3 wherein the protein comprises at least a component of the surface spike protein, and wherein the microbe is SARS-CoV-2.

5. The biological processing apparatus of claim 1 wherein the first fluid tubing is located proximate to a first tool port of the first processing apparatus and a second tool port of the second processing apparatus wherein when the first toolpod containing a first processing apparatus and the second toolpod containing a second processing apparatus are advanced into their operating position the first fluid tubing resides at least in part in the primary cleanspace.

6. The biological processing apparatus of claim 5 further comprising:
a means of chemically sterilizing at least a first tube within the first fluid tubing; and
a means of sterilizing the tool ports and the interconnection when it is in the primary cleanspace.

7. The biological processing apparatus of claim 6 wherein the means of chemically sterilizing the first tube comprises a fluid solution comprising ozone.

8. The biological processing apparatus of claim 6 wherein the means of chemically sterilizing the first tube comprises a fluid solution comprising chlorine.

9. The biological processing apparatus of claim 6 wherein the means of chemically sterilizing the first tube comprises a fluid solution comprising steam.

10. The biological processing apparatus of claim 1 further comprising a shroud surrounding a first tool port of the first toolpod, wherein the shroud creates a sealing surface to a fabricator wall.

11. The biological processing apparatus of claim 1 further comprising a shroud surrounding the periphery of a first tool port of the first toolpod, the first fluid tubing between the first toolpod and the second toolpod, and a second tool port of the second toolpod.

12. The biological processing apparatus of claim 1 further comprising a modelling system, wherein the modelling system is configured to produce a first digital model which is used to control at least a first processing apparatus of the first toolpod, wherein the first processing apparatus controls equipment to create one or more of a tissue support matrix and a printed deposit of cellular and molecular material.

13. The biological processing apparatus of claim 1 further comprising a second substrate with a multitude of printing elements arrayed thereupon, wherein the printing elements are capable of emitting a fluid comprising at least a first cell to a region within a third processing apparatus based upon a final three-dimensional model.

14. The biological processing apparatus of claim 13 further comprising a microfluidic processing system to process cellular and chemical material and deliver a product to the printing elements.

15. The biological processing apparatus of claim 1 further comprising a second substrate, wherein the second substrate comprises at least a first bioreactor chamber, at least a first purification element, at least a first valve, at least a first identification element, and at least a first chemical sensor.

16. The biological processing apparatus of claim 15 wherein the second substrate further comprises an artificial intelligence chip.

17. A method of forming a tissue layer comprising:
configuring a tissue engineering apparatus comprising:
a cleanspace fabricator, wherein the cleanspace fabricator is configured to utilize at least a first substrate comprising tissue layers, wherein the cleanspace fabricator maintains both a particulate cleanliness as well as a biological sterility cleanliness, wherein the cleanspace fabricator comprises at least a first processing apparatus and a second processing apparatus deployed along a periphery of the cleanspace fabricator, and wherein the cleanspace fabricator comprises automation to move one or more of the first substrate and the first processing apparatus within a primary cleanspace of the cleanspace fabricator;
a first and a second toolpod, wherein the first and second toolpod
comprise at least a first fluid tubing that flows between the first and second toolpod;
a modelling system, wherein the modelling system is configured to produce a first digital model which is used to control at least the first processing apparatus, wherein the first processing apparatus controls equipment to create one or more of a tissue support matrix and a printed deposit of cellular and molecular material;
wherein the first processing apparatus comprises a second substrate with a multitude of printing elements arrayed thereupon, wherein the printing elements are capable of emitting a fluid comprising at least a first cell to a region within the first processing apparatus based upon a final three-dimensional model; and
wherein the first processing apparatus further comprises a microfluidic processing system to process cellular and chemical material and deliver a product to the printing elements;
placing a first sample of cells within the cleanspace fabricator;
moving a first portion of the sample of cells into a bioreactor;
incubating the cells in the bioreactor;
flowing a fluid comprising the first portion of the sample of cells from the bioreactor into a cellular washing system through the first fluid tubing;
concentrating the sample of cells in a concentrating system;
placing the first substrate within the cleanspace fabricator;
creating a final digital model, wherein the final digital model represents a three-dimensional model for depositing of cellular material;
forming one or more individual printing system elements;
aligning the one or more individual printing system elements in space relative to the first substrate; and
printing cells from the concentrated sample of cells upon the first substrate, using location control signals that are based upon the final digital model.

18. The method of claim 17 further comprising:
genetically modifying DNA or RNA of cells of the first sample, wherein the genetic modification renders the cell to be an omnipotent stem cell; and
sorting the omnipotent stem cells from other cells to create a second stock of cells.

19. The method of claim 17 wherein a product of printing the first sample of cells forms a neuron to electronics electrical interface.
